(19) United States
(12) Patent Application Publication
(10) Pub. No.: US 2008/0299097 A1
(43) Pub. Date: Dec. 4, 2008

(54) ISOLATED POPULATION OF RAPIDLY PROLIFERATING MARROW STROMAL CELLS FOR ENHANCED IN VIVO ENGRAFTMENT

(75) Inventors: Darwin J. Prockop, New Orleans, LA (US); Ryang Hwa Lee, New Orleans, LA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Tulane University Health Sciences Center, New Orleans, LA (US)

(21) Appl. No.: 11/937,089

(22) Filed: Nov. 8, 2007

Related U.S. Application Data
(60) Provisional application No. 60/864,847, filed on Nov. 8, 2006.

(51) Int. Cl. A61K 35/12; A61P 43/00

(52) U.S. Cl. 424/93.21

(57) ABSTRACT

Multipotent stromal cells “MSCs” have been described as consisting of at least two populations of cells, rapidly self-renewing stem cells (RS-MSCs), and larger, slowly replicating cells (mMSCs). The present invention provides methods for enhancing engraftment of MSCs in vivo by administering an enriched fraction of RS-MSCs that express certain cell surface markers.

This application claims priority to U.S. Provisional Application No. 60/864,847, filed Nov. 8, 2006, which is incorporated herein by reference in its entirety.

The present invention was made in part with support from grants obtained from the National Institutes of Health. The federal government may have rights in the present invention.

### FIELD OF THE INVENTION

The present invention generally relates to the identification and characterization of classes of small rapidly self-renewing stem cells (RS-MSCs) that show increased engraftment when administered in vivo.

### BACKGROUND OF THE INVENTION

Bone marrow contains at least two kinds of stem cells, hematopoietic stem cells and stem cells for non-hematopoietic tissues (1-27). Stem cells for non-hematopoietic tissues have been defined by their ability to adhere to plastic and are sometimes referred to as “plastic adherent stem/progenitor cells,” “fibroblastoid colony forming units,” “mesenchymal stem cells,” “marrow stromal cells,” “multipotential stromal cells,” and most recently, “multipotent stromal cells” (MSCs). MSCs are easily isolated from a small aspirate of bone marrow, and readily generate single-cell derived colonies (1, 2, 5, 18, 21, 25, 27). Single-cell derived colonies of MSCs can be expanded through as many as 50 population doublings in about 10 weeks (25).

MSCs can differentiate into osteoblasts, adipocytes, chondrocytes (1, 13), myocytes (9), astrocytes, oligodendrocytes, and neurons (17, 23, 26, 27). For these reasons, MSCs are currently being tested for their potential use in cell and gene therapy of a number of human diseases (22, 24). MSCs are attractive candidates for cell and gene therapies because they are readily obtained from the patient to be treated and therefore do not generate immune responses. Also, MSCs have a limited tendency to produce tumors, a prominent feature of embryonic stem cells.

As early passage MSCs and MSCs passaged at very low plating densities expand in culture, they generate single-cell derived colonies that contain two populations of cells termed RS-MSCs, a population of small and rapidly self-renewing MSCs, and mMSCs, a population of larger MSCs that arise after MSCs have been cultured in vivo (3, 40). RS-MSCs have also been called “RS-cells,” and “small rapidly self-renewing stem cells,” while mMSCs have also been called “SR-cells,” “SR-MSCs,” and “large, more mature cells” (3, 40).

Although cultures of MSCs have been studied extensively for over 30 years (1), rigorous standards for characterizing and isolating homogenous populations of cells have not emerged. Therefore, it has been difficult to compare populations of MSCs prepared using different protocols, or to ensure that a population of cells is relatively homogenous. These shortcomings have increased significance as clinical trials using cultures of MSCs are underway (22, 24).

While the recent characterization of populations of RS-MSCs and mMSCs (40) has facilitated the in vivo study of particular populations of MSCs, the present invention addresses the need for defined populations of MSCs, and provides methods for the enhanced engraftment of MSCs in vivo using such defined populations.

### SUMMARY OF THE INVENTION

It has now been demonstrated that RS-MSCs engraft more efficiently than mMSCs when administered to immunodeficient mice. It has also been demonstrated that RS-MSCs can be further enriched for RS-MSCs that express certain surface polypeptides, namely CXCR4 and CX3CR1, and that these cells exhibit enhanced capabilities for engraftment when administered in vivo.

Accordingly, the present invention provides a method for enhancing the engraftment of MSCs in an individual. In one embodiment, an individual is administered a population of MSCs that are enriched for RS-MSCs. In a preferred embodiment, the enriched population of MSCs express at least one of the polypeptides selected from the group consisting of CXCR4 and CX3CR1. Preferably, the enriched population expresses both CXCR4 and CX3CR1.

MSCs can be isolated from tissues including bone marrow, peripheral blood, umbilical cord blood, and synovial membrane. In one preferred embodiment the MSCs are isolated from bone marrow.

MSCs for administration can be isolated from the individual to be treated, i.e. autologous, or isolated from another individual, i.e. allogeneic. For allogeneic MSCs, it is preferred that the donor and the individual to be treated are HLA compatible.

The MSCs can be administered by infusion, including intravenous infusion, systemic infusion, intra-arterial infusion, intracoronary infusion, and intracardiac infusion. The MSCs can also be administered by intravenous injection or injection directly to the site of intended activity.

### BRIEF DESCRIPTION OF THE DRAWINGS

FIGS. 1A-1D show an analysis of human MSCs after five days in culture by FACS and cell cycle assay. FIG. 1A shows a re-assay of forward scatter/side scatter of RS-MSCs after sorting for low forward scatter/low side scatter. FIG. 1B shows a re-assay of mMSCs after sorting for high forward scatter/high side scatter. FIG. 1C shows a cell cycle analysis of the cells shown in FIG. 1A (RS-MSCs). FIG. 1D shows a cell cycle analysis of the cells shown in FIG. 1B (mMSCs).

FIG. 2 shows an experimental scheme for detection of human MSCs with allele-specific SNPs. The levels of engraftment were assayed by real-time PCR of Alu sequences.

FIG. 3 shows SNP region sequences and SNP-specific primers used to define the genotype of the engrafted human MSCs.

FIGS. 4A-4D show graphs of real time PCR assays for engrafted human MSCs. FIG. 4A shows results of real time PCR with forward and reverse primers for the A/A allele; samples contained 200 ng of mouse DNA, 1 ng of the G/G allele and 1 pg to 1 ng of the A/A allele. FIG. 4C shows results of real time PCR with forward and reverse primers for the G/G allele; samples contained 200 ng mouse DNA, 1 ng of the A/A allele and 1 pg to 1 ng of the G/G allele. FIG. 4B shows the same sample as shown in FIG. 4A, but RNA was amplified with primers for the G/G allele. FIG. 4D shows the same sample as shown in FIG. 4B, but RNA was amplified with primers for the A/A allele.

FIGS. 5A-5C show representative results from an engraftment assay after intravenous infusion of 1:1 mixtures of 2.5×10⁵ RS-MSCs and mMSCs from donors with different A/A and G/G alleles. FIG. 5A shows a schematic of brain regions assayed. FIG. 5B shows a representative result of a competitive SNP assay in which RS-MSCs engraft better than mMSCs. Genomic DNA was isolated from brain section D (see FIG. 5A). FIG. 5C shows the results of an immunohistochemistry analysis of section D with an antihuman nuclear antigen antibody (magnification=600×).

FIGS. 6A-6C show representative results from an engraftment assay after direct injection of RS-MSCs and mMSCs into the hippocampus of immunodeficient mice. Engraftment in the hippocampus, section D, is analyzed.

FIG. 6A shows a schematic of the brain regions assayed. FIG. 6B shows a representative result of a competitive SNP assay, in which RS-MSCs engraft in the hippocampus. Genomic DNA was isolated from brain section D (see FIG. 6A).

FIG. 6C shows results of an immunohistochemistry analysis of section D with an antihuman nuclear antigen antibody (magnification=600×).

FIGS. 7A and 7B show tables describing engraftment of MSCs in vivo. FIG. 7A shows values for eight different mice after injection of 1:1 mixture of 2.5×10⁵ RS-MSCs and mMSCs. FIG. 7B shows values for engraftment in brain after intracranial injections of either 2.5×10⁵ RS-MSCs from one donor or a 1:1 mixture of 5×10⁴ RS-MSCs from one donor and 5×10⁴ mMSCs from a second donor. Data are presented as % of injected cells.

FIGS. 8A and 8B show analysis of the migration of RS-MSCs and mMSCs in response to neurospheres (FIG. 8A), and analysis of expression of various chemokines (FIG. 8B). FIG. 8A shows that RS-MSCs migrate better than mMSCs in response to neurospheres. Migration was followed by fluorimetry of the underside of opaque inserts in transwells. Data are expressed as mean and range of 2 values. FIG. 8B shows an RT-PCR assay of total RNA for chemokine receptors. Lane 1: RS-MSCs. Lane 2: mMSCs. Lane 3: Unsorted MSCs. The results show that RS-MSCs express higher levels of CX3CR1 and CXCR4 than mMSCs.

FIGS. 9A-9H show an analysis of CXCR4 and CX3CR1 as epitopes on MSCs. FIG. 9A shows flow cytometry of unsorted MSCs with anti-CXCR4-PE. About 8% of the unfractionated cells expressed CXCR4, and the positive cells are primarily low in forward scatter (FIG. 9A). FIG. 9B shows real-time RT-PCR assay of CXCR4 in RS-MSCs, mMSCs, and unsorted MSCs. FIG. 9C shows real-time RT-PCR assay of GAPDH as a loading standard. FIG. 9D shows immunocytochemistry of RS-MSCs and mMSCs cultured in chambered slides and stained with antibody to CXCR4 and DAPI (magnification=400×). FIG. 9E shows flow cytometry of unsorted MSCs with anti-CX3CR1-FITC. About 3% of the unfractionated cells expressed CX3CR1, and the positive cells are primarily low in forward scatter (FIG. 9E). The positive cells are primarily low in forward scatter. FIG. 9F shows real-time RT-PCR assay of CX3CR1 in RS-MSCSs, mMSCs, and unsorted MSCs. FIG. 9G shows real-time RT-PCR assay of GAPDH. FIG. 9H shows immunocytochemistry of RS-MSCs and mMSCs cultured in chambered slides and stained with antibody to CX3CR1 and DAPI (magnification=400×).

FIGS. 10A and 10B show migration of RS-MSCs and mMSCs. FIG. 10A shows migration of RS-MSCs and mMSCs induced by SDF-1 and fractalkine. The bottom wells contained either 20% fetal calf serum, serum-free medium, 50 ng/mL SDF-1, 50 ng/mL SDF-1 plus 10 μg/mL anti-CXCR4, 10 ng/mL fractalkine, or 10 ng/mL fractaline plus 5 μg/mL anti-CX3CR1. Migration was assayed by photomicrograph of the underside of the opaque inserts (magnification=200×). Data are expressed as mean and range of 2 values. FIG. 10B shows migration of RS-MSCs and mMSCs in response to neurospheres. Assays and values are as in FIG. 10A, except SDF-1 and fractalkine were replaced with about 1×10⁵ neural stem cells from neurospheres. Data are expressed as mean and range of 2 values.

### DETAILED DESCRIPTION

This invention is based in part on the discovery that RS-MSCs engraft more efficiently than mMSCs in vivo. In addition, it has now been shown that RS-MSCs that express at least one of CXCR4 and CX3CR1 have increased capacity for engraftment. Accordingly, the present invention provides methods for enhancing engraftment of MSCs in an individual by administering RS-MSCs that express at least one of CXCR4 and CX3CR1. Preferably, the RS-MSCs express CXCR4 and CX3CR1.

As used herein, “CXCR4” is an alpha-chemokine receptor specific for stromal-derived-factor-1 (SDF-1 also called CXCL12), a molecule with potent chemotactic activity for lymphocytes. CXCR4 is also known to those of skill in the art as “neuropeptide y receptor y3” (npy3r), “fusin,” “D2s201e,” “Leukocyte-derived seven-transmembrane-domain receptor” (lestr), “Seven-transmembrane-segment receptor” (spleen), “Hm89,” “Lipopolysaccharide-associated protein 3” (lap3), or “Lps-associated protein 3” (OMIM: 162643).

As used herein, “CX3CR1” is a chemokine receptor specific for fractalkine (also called CX3CR1). CX3CR1 is also known to those of skill in the art as “fractalkine receptor,” “G protein-coupled receptor 13” (gpr13), or “G protein-coupled receptor v28” (v28) (OMIM: 601470).

### Multipotent Stromal Cells (MSCs)

Bone marrow contains at least two types of stem cells, hematopoietic stem cells (HSCs) and stem cells for non-hematopoietic tissues, referred to here as multipotent stromal cells (MSCs). These plastic adherent stem/progenitor cells isolated from bone marrow were initially referred to as fibroblastoid colony forming units, then in the hematological literature as marrow stromal cells, then as mesenchymal stem cells, and most recently as multipotential or multipotent stromal cells (MSCs); these cells have also been referred to mesenchymal stem cells, bone marrow stromal cells, or simply stromal cells (13). MSCs are sometimes referred to as mesenchymal stem cells because they are capable of differentiating into multiple mesodermal tissues, including bone (8), cartilage (41), fat (8) and muscle (9).

In one embodiment, a preferred population of MSCs is an enriched, or substantially homogenous, population of RS-MSCs. RS-MSCs are described in detail in U.S. Pat. No. 7,056,738, which is incorporated herein by reference in its entirety. In particular, U.S. Pat. No. 7,056,738 provides a population of RS-MSCs that may be differentiated from mMSCs, particularly at column 2, line 58 to column 3, line 51, and Table 1. These particular sections of U.S. Pat. No. 7,056,738 are also incorporated herein by reference.

RS-MSCs and particularly RS-MSCs that express at least one of the polypeptides CXCR4 and CX3CR1 show improved engraftment upon transplantation into immunodeficient mice when compared to a population of mMSCs.

MSCs can give rise to cells of all three germ layers, depending on conditions (23, 28, 38, 42, 30). For example, in vivo evidence indicates that unfractionated bone marrow-derived cells as well as pure populations of MSCs can give rise to epithelial cell-types including those of the lung (29, 43). Similarly, differentiation into neuron-like cells expressing neuronal markers has been reported (27, 44, 45). Under physiological conditions, MSCs are believed to maintain the architecture of bone marrow and regulate hematopoiesis with the help of different cell adhesion molecules and the secretion of cytokines, respectively (7).

MSCs have been used with encouraging results for transplantation in animal disease models including osteogenesis imperfecta (14), parkinsonism (46), spinal cord injury (26, 47) and cardiac disorders (48, 49). Promising results also have been reported in clinical trials for osteogenesis imperfecta (50, 51) and enhanced engraftment of heterologous bone marrow transplants (52, 53). Several studies have shown that engraftment of MSCs is enhanced by tissue injury (16, 54).

MSCs are easily isolated from a small aspirate of bone marrow, and readily generate single-cell derived colonies. MSCs grown out of bone marrow cell suspensions by their selective attachment to tissue culture plastic can be efficiently expanded (17, 25) and genetically manipulated (46).

In general, the isolation and characterization of the RS-MSCs of the present invention involves the following steps: 1) isolation of MSCs; 2) culture and expansion of MSCs in vitro, and 3) characterization and/or separation or enrichment of a substantially homogenous population of RS-MSCs from other isolated MSCs based on forward and side scatter properties after FACS analysis, and/or the expression of selected surface polypeptides.

### Isolation of MSCs

The RS-MSCs of the invention are isolated from other cells of their tissue of origin. The term “isolated” as used herein means that the cells are substantially purified from other cells, cellular components, and/or extracellular materials present in the tissue from which the MSCs are obtained. For example, bone marrow-derived MSCs are substantially purified from the other cells, such as hematopoietic stem cells, which are present in the bone marrow. The isolated population of RS-MSCs of the present invention are substantially purified or isolated from other MSCs, such as mMSCs. The MSCs of the invention are not differentiated, but remain multipotential.

RS-MSCs of the invention can be isolated from different tissue sources, including bone marrow, peripheral blood, umbilical cord blood, and synovial membrane. Other sources of MSCs include, but are not limited to, embryonic yolk sac, placenta, fat, fetal and adolescent skin, and muscle tissue. In certain preferred embodiments, MSCs can be isolated from bone marrow.

A first step in isolating RS-MSCs is the isolation of MSCs. Methods for isolating MSCs according to the invention are known in the art. Methods for isolating MSCs from bone marrow are described for example in U.S. Pat. No. 5,486,359, as well as U.S. Patent Publication Nos. 2003/0003090, 2004/0235166, 2005/0084494, and 2004/0235165, which are incorporated herein by reference in their entirety. Methods for isolating MSCs from umbilical cord blood are described in (55), which is incorporated herein by reference in its entirety. Methods for isolating MSCs from synovial membrane are described for example in (56), which is incorporated herein by reference in its entirety. In general, techniques for the rapid isolation of MSCs include, but are not limited to, leucopheresis, density gradient fractionation, immunoselection, differential adhesion separation, and the like.

One preferred method for isolating MSCs involves collecting bone marrow aspirates, for example from the iliac crest, isolating the mononuclear cells on a density gradient, and plating the cells in culture to allow removal of non-adherent cells; the plastic-adherent cells which remain are MSCs. For example, non-adherent cells can be removed by removing the culture medium and washing the adherent cells after 24 hours in culture. This method is described in detail, for example, in U.S. Patent Publication Nos. 2003/0003090, 2004/0235166, 2005/0084494, and 2004/0235165, which are incorporated herein by reference in their entirety. Bone marrow cells may be obtained from iliac crest, femora, tibiae, spine, rib, or other medullary spaces.

Another preferred method for isolating a heterogenous population of MSCs is described in detail at column 4, lines 1-21 of U.S. Pat. No. 7,056,738, which is incorporated herein by reference in its entirety. In this method, the population of MSCs comprise both RS-MSCs, a population of small and rapidly self-renewing MSCs and mMSCs, a population of larger MSCs. The methods described in U.S. Pat. No. 7,056,738 include isolating nucleated cells from bone marrow aspirates and plating in a culture dish with complete culture medium. After 1 to 2 days, the cells are washed, and the viable plastic adherent cells are harvested with a mixture of trypsin and EDTA (ethylenediaminetetraacetic acid) (e.g., 0.25% trypsin and 1 mM EDTA) or EDTA alone.

An initial round of selection or immunoselection may also be used to isolate MSCs, generally, using monoclonal or polyclonal antibodies raised against surface antigens expressed by bone marrow-derived MSCs (e.g. human MSCs “hMSCs”). For example, U.S. Pat. No. 6,387,367 describes the use of monoclonal antibodies SH2, SH3 or SH4; the SH2 antibody binds to endoglin (CD105), while SH3 and SH4 bind CD73. A stro-1 antibody is described in (57).

MSCs may be derived from any animal, including, but not limited to a rodent, a horse, a cow, a pig, a dog, a cat, a non-human primate, and a human.

MSCs of the invention can be autologous, allogeneic or xenogeneic. The term “autologous” as used herein means that the transplant is derived from the cells, tissues or organs of the recipient. The term “allogeneic” as used herein means that the transplant is derived from cells, tissues, or organs that are of the same species as the recipient but antigenically distinct. The term “xenogeneic” as used herein means that the transplant is derived from the cells, tissues, or organs originating from a different species.

### Culture and Expansion of MSCs In Vitro

In order to enrich for a substantially homogenous population of RS-MSCs, the plastic adherent cells, which are a mixture of RS-MSCs and mMSCs, are plated at low density (e.g. 3 cells/cm²-150 cells/cm², preferably 100 cells/cm²) in 175-cm² culture dishes in complete culture medium. Medium is replaced every 2 to 3 days, and cells are harvested at about day 4 to day 10, preferably day 5 to day 7, but before they reach confluency so that the cultures retain a special population of small, spindle-shaped cells referred to as RS-MSCs.

In one embodiment, the cells are harvested with EDTA/trypsin at about day 5 to day 7 when they reach about 50% to 70% confluency. In a preferred embodiment, the cells are harvested with EDTA alone. As used herein, “density” and “confluency” refer to the state of cells in cell culture (e.g. in vitro). Confluency refers to the coverage or proliferation that the cells are allowed over or throughout the culture medium. This observation is often related to the color of the media supporting the cells (i.e. rate of consumption), the number of dead, floating cells that have not attached to the plate, and the volume of tissue culture dish that is not occupied with adherent cells. The measurement of confluency is typically non-quantitative and is routine to those of skill in the art. Optimum density for the enriched, substantially homogenous population of RS-MSCs of the present invention is 50% to 80%, preferably 50% to 70%, 50% to 65%, 50% to 60%, 50% to 55% confluent.

The population RS-MSCs isolated at this stage are substantially homogenous, and it is shown here that this population consists of RS-MSCs engraft more efficiently into immunodeficient mice than mMSCs that are found in more confluent cultures.

RS-MSCs may also be isolated on the basis of size and/or granularity. In one embodiment, the MSCs are separated on the basis of forward scatter (FS) and side scatter (SS) of light using fluorescence activated cell sorter (FACS). Forward scatter provides an approximate value for cell size, and side scatter provides an approximate value for cellular complexity, or granularity. In a preferred embodiment, the populations of RS-MSCs of the present invention are among the cells that are found in the lower left quadrant of the plot of FS/SS (approximately 20% of the total cells). On the other hand, mMSCs are found in the upper right quadrant of the FS/SS plot.

MSCs may be frozen following isolation from the bone marrow, and stored for any length of time that does not compromise their function, pluripotency or viability. MSCs can be frozen immediately after isolation, or cultured and expanded after isolation but prior to freezing. MSCs may be enriched for the RS-MSCs of the present invention before or after freezing. Frozen cells may then be thawed and used for administering to mammals in various therapeutic methods.

MSCs of the invention can be maintained in culture media which can be chemically defined serum free media or can be a “complete medium”, such as Dulbecco's Modified Eagles Medium supplemented with 10% serum (DMEM), or alpha-MEM supplemented with 20% serum. Suitable chemically defined serum free media and complete media are well known in the art, see for example U.S. Pat. No. 5,908,782, WO96/39487, and U.S. Pat. No. 5,486,359. Chemically defined medium typically comprises a minimum essential medium such as Iscove's Modified Dulbecco's Medium (IMDM), supplemented with human serum albumin, human Ex Cyte lipoprotein, transferrin, insulin, vitamins, essential and non-essential amino acids, sodium pyruvate, glutamine and a mitogen. These media stimulate multipotent stromal cell growth without differentiation.

RS-MSCs may be cultured under conditions to remove any non-human serum proteins, for example, prior to their administration to humans. Such methods include the use of short-term cultures in human serum or platelet lysate to metabolically remove non-human serum proteins (Spees et al., Molec. Ther. 9:747-756 (2004), see materials and methods on page 5, which is incorporated herein by reference in its entirety).

In certain embodiments, MSCs can be genetically modified prior to administration to the individual. For example, the MSCs can be genetically modified to express a recombinant polypeptide, such as a growth factor, chemokine, or cytokine, or a receptor which binds growth factors, chemokines, or cytokines. The MSCs can also be genetically modified to express a marker protein such as GFP which allows their identification in the recipient.

### Characterization of Isolated MSCs

In one embodiment of the present invention, an isolated population of MSCs, more specifically, an isolated population of RS-MSCs are characterized by the positive expression of certain polypeptides using immunological techniques well known in the art, e.g., antibody techniques such as immunohistochemistry, immunocytochemistry, FACS scanning, immunoblotting, radioimmunoassays, western blotting, immunoprecipitation, and enzyme-linked immunosorbant assays (ELISA).

In one embodiment, an enriched population of RS-MSCs is provided. The isolated RS-MSCs are characterized by the expression of at least one of the polypeptides selected from the group consisting of CXCR4 and CX3CR1. In one embodiment, the cells co-express CXCR4 and CX3CR1.

“Co-express,” as used herein, refers to the simultaneous detection of two or more molecules, e.g., CXCR4 and CX3CR1, on or in a single cell. Techniques to detect co-expression of CXCR4 and CX3CR1 in cells (e.g., and enriched population of RS-MSCs) are well established. For example, co-expression of CXCR4 and CX3CR1 on or in a cell can be detected by multiple color cytometric analysis. CXCR4 can be detected employing a fluorescein labeled probe and CX3CR1 can be detected employing a Texas red probe. The CXCR4 and CX3CR1 cell surface antigens can be visualized with the aid of a flow cytometer equipped with multiple filters capable of detecting the multiple colors of the fluorescent probes. Techniques to detect the molecules of interest can also include ELISA, RIA, immunoflouresence microscopy and quantitative PCR.

In one embodiment, a method for administering an enriched population of RS-MSCs that express at least one of CXCR4 and CX3CR1 is provided. The enriched population of RS-MSCs is characterized by the expression of at least one of CXCR4 and CX3CR1. The expression may be internal (i.e., inside the cell), or external (i.e., expressed on the surface of the cell). In a preferred embodiment, the polypeptides are internally expressed. One of skill in the art will recognize that a cell may need to be lysed prior to analyzing internal expression of proteins.

### Methods of Administration

RS-MSCs herein described may be administered or transplanted to a mammal. The term “transplanting” as used herein means introducing a cellular, tissue or organ composition into the body of a mammal by any method known in the art, or as indicated herein. The composition is a “transplant,” and the mammal is the recipient.

The transplant and recipient may be syngeneic, allogenic, or xenogeneic. The term “syngeneic” as used herein means that the transplant is derived from cells, tissues, or organs that are of the same species as the recipient, and antigenically the same or similar enough so as not to illicit an immune response, i.e., that are histocompatible. Syngeneic cells are sometimes referred to herein as “HLA compatible.”

In one embodiment, the animal to which the RS-MSCs are administered is a mammal. The mammal may be a rodent, a horse, a cow, a pig, a dog, a cat, a non-human primate, and a human.

The RS-MSCs can be administered to the individual by a variety of procedures. The MSCs may be administered systemically, such as by intravenous, intraarterial, or intraperitoneal administration, or the MSCs may be administered directly to a tissue or organ such as the pancreas or kidney, for example by direct injection into the tissue or organ.

The RS-MSCs are administered to the individual in a therapeutically effective amount, as described above. In general, the MSCs are administered in an amount from about 1×10⁵ cells/kg to about 1×10⁷ cells/kg. The exact amount of MSCs to be administered is dependent upon a variety of factors, including the age, weight, and sex of the patient, and the extent and severity of the condition being treated.

The RS-MSCs may be administered in conjunction with an acceptable pharmaceutical carrier. For example, the MSCs may be administered as a cell suspension in a pharmaceutically acceptable liquid medium for injection.

It is to be understood that the RS-MSCs may be administered in combination with other therapeutic agents known to those skilled in the art. In one embodiment, the recipient can be administered an agent that suppresses the immune system, such as Tacrolimus, Sirolimus, cyclosporine, and cortisone and other drugs known in the art. See e.g. U.S. Patent Publication No. 2004/0209801. Other immunosuppressive agents which can be used include anti-CD11 antibody.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

All references herein provided are incorporated by reference in their entirety.

### EXAMPLES

### Characterization of RS-MSCs with Enhanced Engraftment Capabilities

In this Example, both mMSCs and RS-MSCs are characterized. The results show that RS-MSCs engraft more efficiently in vivo than mMSCs. The results also show that a population of RS-MSCs that express CXCR4 and CX3CR1 have the potential to migrate and engraft more efficiently in vivo than a population of MSCs without these cellular signatures. Such a finding can guide one in the isolation of MSCs that are clinically relevant.

We first developed a sensitive polymerase chain reaction-based single nucleotide polymorphism (PCR-SNP) assay for the competitive engraftment of mixtures of MSCs from 2 different human donors. The assay demonstrates marked differences in the engraftment of human RS-MSCs and mMSCs after either intravenous or intracranial infusion into immunodeficient mice. Moreover, we identify two markers, CXCR4 and CX3CR1 that are expressed on a population of RS-MSCs, which engrafts more efficiently in vivo. In addition, we show that RS-MSCs migrate more rapidly to murine neurospheres than mMSCs and their migration is selectively decreased by neutralizing antibodies to CXCR4 and CX3CR1.

### A. Isolation and Culture of Human MSCs

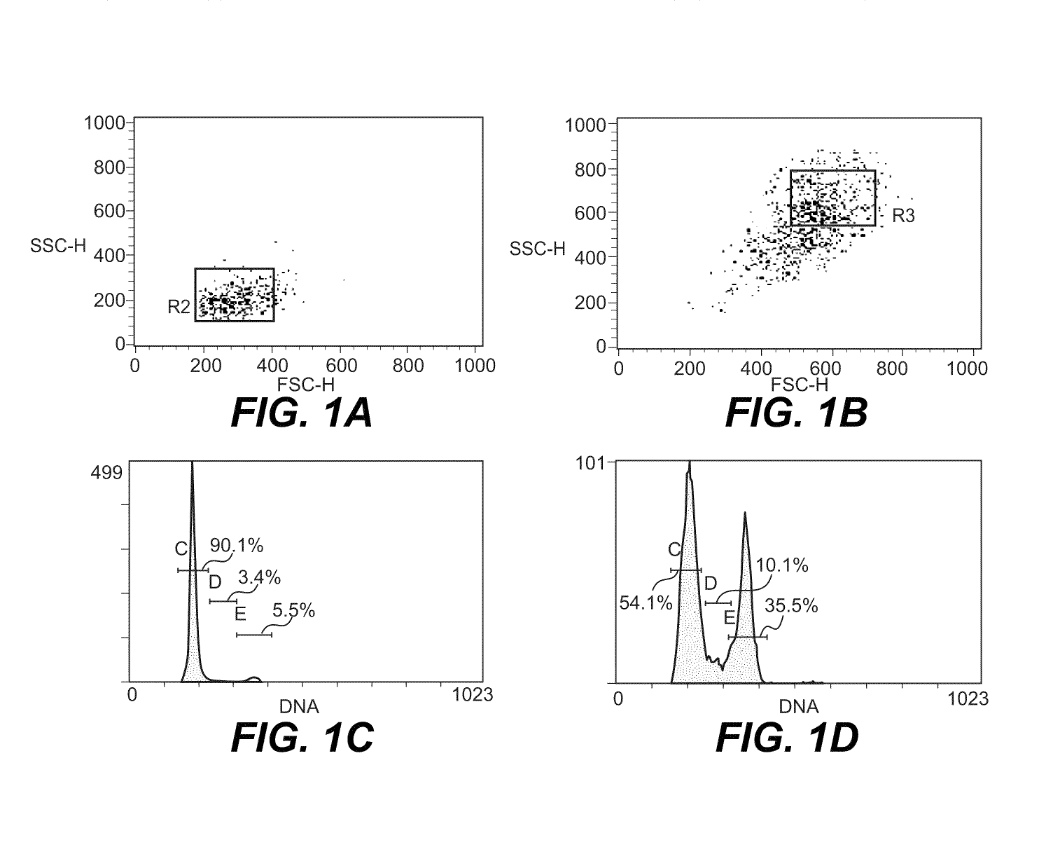

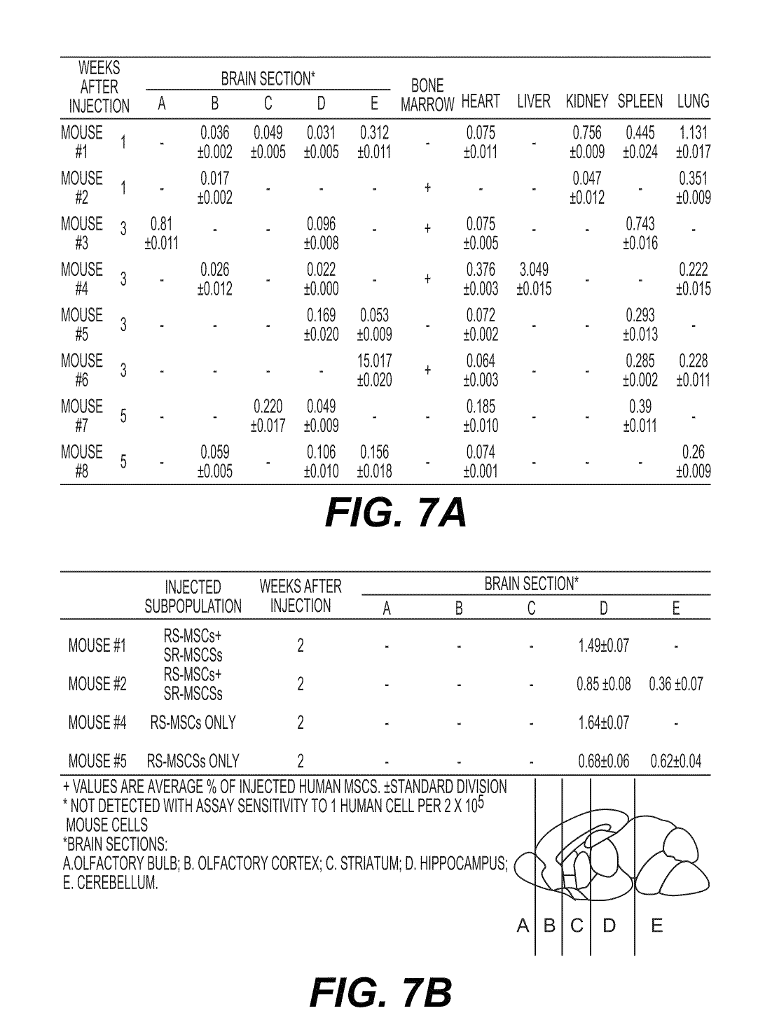

Frozen vials of human MSCs (hMSCs) from 2 healthy donors, prepared as described previously (32, 34), were obtained from the Tulane Center for Distribution of Adult Stem Cells (Center for Gene Therapy, Tulane University Health Sciences Center, New Orleans, La.). The MSCs were prescreened to establish that the cells from the donors were distinguishable on the basis of SNPs in the first intron of the COL1A2 gene (Table 2 and FIG. 7). To expand the MSCs, a frozen vial of about 10⁶ MSCs from Passage 2 was thawed, plated in a 175-cm² dish, and incubated in 25 mL complete culture medium (CCM) composed of alpha-minimum essential medium (alpha-MEM; GIBCO/BRL, Grand Island, N.Y.); 20% fetal bovine serum (FBS; lot-selected for rapid growth of MSCs; Atlanta Biologicals, Miami, Fla.); 100 U/mL penicillin; 100 μg/mL streptomycin; and 2 mM L-glutamine (GIBCO/BRL). After 1 to 2 days, the adherent cell layer was washed with phosphate buffered saline (PBS), and viable adherent cells were harvested with 0.25% trypsin and 1 mM EDTA (ethylenediaminetetraacetic acid) for 5 minutes at 37° C. The cells were expanded by plating at initial densities of about 100 cells/cm² in 175-cm² dishes and in the CCM. The medium was replaced every 2 to 3 days. The cultures were lifted with EDTA/trypsin after they reached about 70% confluency in 5 to 7 days. Cell numbers were counted with a hemocytometer. To isolate MSCs enriched for RS-MSCs, MSCs from 5- to 7-day cultures were separated on the basis of forward scatter (FS) or side scatter (SS) of light using FACS Vantage SE flow cytometer (Becton Dickinson, San Jose, Calif.). The samples were sorted by gating for about 10-20% of the events in the lower left quadrant (RS-MSCs) and about 10-20% of the cells in the upper right quadrant of the plot of FS/SS (mMSCs; FIG. 1).

### B. Cell Cycle Profile

2.5-5.0×10⁵ MSCs in 100 μL PBS were mixed with 100 μL lysis buffer (DNA-Prep LPR; Beckman-Coulter, Fullerton, Calif.). The sample was gently vortexed and immediately afterward 500 μL of a propidium iodide solution containing a lysis buffer and a staining reagent (DNA-Prep Stain Reagent; Beckman-Coulter) was added. After gently vortexing for 5 seconds, the sample was incubated for 20 minutes at room temperature in the dark. The sample was assayed within 2 hours by flow cytometry (Cytomic FC 500; Beckman-Coulter), and cell cycle status was calculated (ModFit 3.0; Beckman-Coulter).

### C. Intravenous and Cerebral Infusions

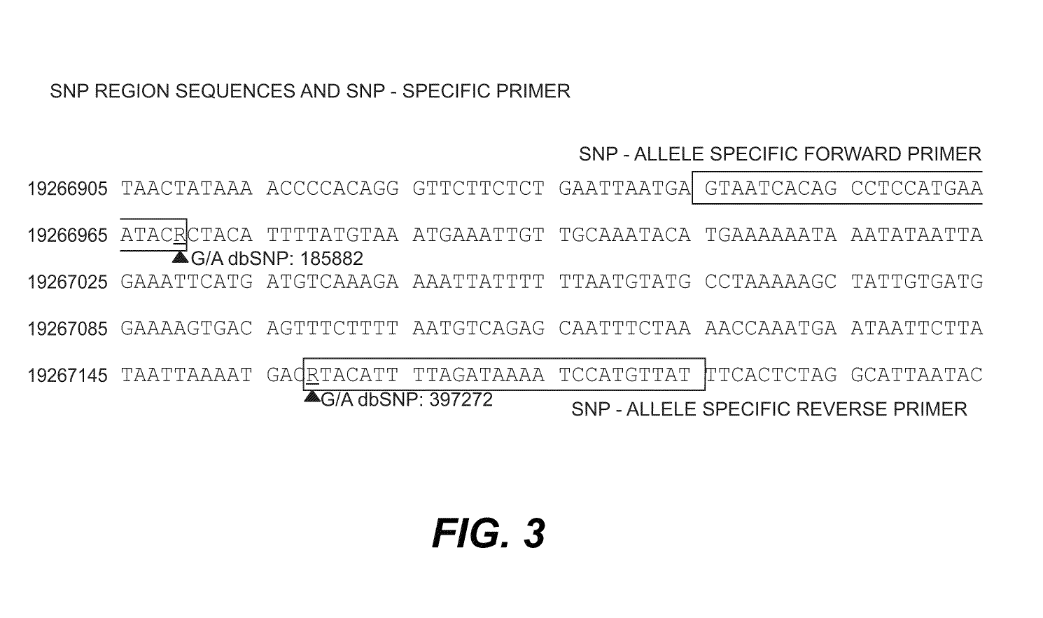

For intravenous infusion, 5- to 6-week-old severe combined immunodeficient (SCID)/Beige mice (Charles River, Wilmington, Mass.) were anesthetized with intraperitoneal injection of 80 mg/kg ketamine and 8 mg/kg xylazine. Tails were immersed in warm water for about 5 minutes and 200 μL of cell suspension in PBS was slowly infused into a tail vein with a 27-gauge needle. The cell suspension was a 1:1 mixture of 2.5×10⁵ RS-MSCs from one human donor and 2.5×10⁵ mMSCs from a second donor whose cells could be distinguished by the presence of a SNP in the COL1A2 gene (Table 2 and FIG. 3). Prior to injection, the cells were maintained at 4° C. and gently resuspended with a pipette to ensure they did not aggregate.

For cerebral infusions, 5- to 6-week-old SCID/Beige mice were anesthetized with ketamine/xylazine and infused with either 100,000 RS-MSCs from one donor or with 1:1 mixtures of 50,000 RS-MSCs from one donor and 50,000 mMSCs from the second donor that could be distinguished by SNPs. The cells were injected through a 30-gauge needle over 5 minutes into the hippocampus of one hemisphere at −2.3 mm posterior, −2.0 mm lateral, and −2.5 mm ventral to bregma. The bevel of the needle was directed caudally.

### D. Assays for Alu and SNPs by Real-Time PCR

For PCR assays, the mice were sacrificed and genomic DNA from tissues was extracted using DNeasy tissue kit (Qiagen, Chatsworth, Calif.). The amount of DNA was assayed first by absorbance and then by real-time PCR assays for the mouse albumin gene to normalize genomic DNA. PCR assays were performed in a volume of 50 μL that contained 25 μL Universal PCR Master Mix (Applied Biosystems, Foster City, Calif.), 900 nM each of the forward and reverse primers, 250 nM TaqMan probe, and 200 ng target template. Reactions were incubated at 50° C. for 2 minutes for optimal uracil-N-glycosylase (UNG) enzyme activity and at 95° C. for 10 minutes to activate AmpliTaq Gold enzyme, followed by 40 cycles of 95° C. for 15 seconds and 60° C. for 1 minute. Standard curves were generated by serially diluting human genomic DNA prepared from MSCs into samples containing 200 ng genomic DNA from mouse brain.

Primers for the mouse albumin gene were designed to amplify a specific intron sequence of the single copy serum albumin variant Alb1 on chromosomes (NCBI; Mus musculus genome view (39). The mouse albumin forward primer was 5′-GAA AAC CAG GCG ACT ATC TCC A-3′ (SEQ ID NO:1); mouse albumin reverse primer was 5′-TGC ACA CTT CCT GGT CCT CA-3′ (SEQ ID NO:2). PCR assays for the mouse albumin gene were performed using SYBR Green PCR Master Mix (Applied Biosystems). The sequence of the PCR primers and the probe used for detection of human Alu repetitive sequences (35) were as follows: Alu forward; 5′-CAT GGT GAA ACC CCG TCT CTA-3′ (SEQ ID NO:3); Alu reverse; 5′-GCC TCA GCC TCC CGA GTA G-3′ (SEQ ID NO:4); TaqMan probe; 5′-FAM-ATT AGC CGG GCG TGG TGG CG-TAMRA-3′ (SEQ ID NO:5) (Applied Biosystems). To express the results as human cells per organ, the detected level of human DNA in 200 ng mouse DNA was multiplied by total DNA per organ extracted with phenol/chloroform and assayed by absorbance. Assuming a value of 5 pg DNA per cell (59) the cellular contents were as follows: brain, 2.59×10⁸±0.43 (n=5); heart, 4.21×10⁷±1.56 (n=5); liver, 1.32×10⁹±0.605 (n=4); kidney, 6.14×10⁸±1.00 (n=5); spleen, 1.90×10⁸±0.797 (n=5); and lung, 2.19×10⁸±0.56 (n=5).

To assay SNPs, sequences in the first intron of the human COL1A2 gene region (Table 2 and FIG. 3) were first amplified with primers flanking the SNPs (36). The PCRs were performed in a volume of 50 μL and contained 1 U/μL recombinant Taq DNA polymerase (Invitrogen, Carlsbad, Calif.), 1× PCR buffer, 0.2 mM each dNTP, 1.5 mM MgCl₂, and 500 nM of each primer. The COL1A2 forward primer was 5′-CAT CCA CAC ACA TGC ACA GA-3′ (SEQ ID NO:6); the COL1A2 reverse primer was 5′-TTT CCC CTT TGT TGT TTC CA-3′ (SEQ ID NO:7). The amplification conditions were 95° C. for 5 minutes, followed by 25 cycles of 94° C. for 1 minute, 58° C. for 1 minute, and 72° C. for 1 minute. After amplification of COL1A2 region, 2 μL of the PCR reaction was used for SNP assays by real-time PCR. The primers were designed to ensure specificity by introducing a mismatch into the penultimate 3′-base (37). The SNP forward primer for the A/A allele was 5′-GTA ATC ACA GCC TCC ATG AAA TAG A-3′ (SEQ ID NO:8); SNP forward primer for the G/G allele was 5′-GTAATC ACA GCC TCC ATG AAA TAT G-3′ (SEQ ID NO:9); SNP reverse primer for the A/A allele was 5′-ATA ACA TGG ATT TTA TCT AAA ATG TGT-3′ (SEQ ID NO:10); SNP reverse primer for the G/G allele was 5′-ATA ACA TGG ATT TTA TCT AAA ATG TGC-3′ (SEQ ID NO:11). The TaqMan probe was 5′-FAM-TGC CTA AAA AGC TAT TGT GAT GGA AAA GTG ACA GT-TAMRA-3′ (SEQ ID NO:12) (Applied Biosystems). The conditions for amplification were the same as for Alu sequences. All real-time PCR assays were performed in duplicate or triplicate and average values are presented.

### E. Immunohistochemistry and Immunocytochemistry

For immunohistochemistry of brain sections, mice were anesthetized with intraperitoneal injection of 80 mg/kg ketamine and 8 mg/kg xylazine, and perfused through the right atrium with 30 mL PBS followed by 10 mL of 4% paraformaldehyde. The tissues were excised, rinsed with PBS, fixed in 4% paraformaldehyde in PBS fixative overnight at 4° C., transferred to 30% sucrose solution overnight at 4° C., and flash frozen. 5-μm sections were cut in a cryostat. The sections were incubated for 18 hours at 4° C. with antibody to a human-specific nuclear antigen (1:100, mouse anti-human nuclei monoclonal antibody; Chemicon, Temecula, Calif.). The slides were washed 3 times for 5 minutes with PBS and incubated for 1 hour at room temperature with secondary antibody (1:1000, Alexa-594; Molecular Probes, Eugene, Oreg.). The slides were counterstained with DAPI (DAPI; Vector Labs, Burlingame, Calif.). Controls included omitting the primary antibody. Slides were evaluated by epifluorescence (Eclipse 800; Nikon, Melville, N.Y.) using a 60× objective.

For immunocytochemistry of cells, RS-MSCs isolated by fluorescence-activated cell sorting (FACS) were plated at initial densities of about 1000 cells/cm² in a slide chamber (LAB-TEK II chamber slide; Nalge Nunc International, Rochester, N.Y.). After incubation for 1 day, the cultures were rinsed with PBS and fixed in 4% paraformaldehyde in PBS for 20 minutes at room temperature. The slide chambers were incubated for 18 hours at 4° C. with antibody to CXCR4 (1:200; Chemicon) and CX3CR1 (1:200; Abcam, Cambridge, Mass.). The slides were washed 3 times for 5 minutes with PBS and incubated for 1 hour at room temperature with secondary antibody (1:1000, Alexa-594; Molecular Probes). The slides were counterstained with DAPI. Controls included omitting the primary antibody. Slides were evaluated by epifluorescence (Eclipse 800; Nikon) using a 40× objective. Images were analyzed using SPOT-RT imaging software (Diagnostic Instruments, Sterling Heights, Mich.).

### F. Migration Assays

Isolated RS-MSCs and mMSCs were labeled by incubation at 37° C. for 30 minutes in alpha-MEM containing a cellular dye (Tracker Green CMFDA; Molecular Probes) and washed 3 times by centrifugation with PBS. Migration assays were carried out in a 24-well transwell using opaque inserts with 8-μm pores (HTS FluoroBlok 24-Multiwell Insert System; BD Biosciences, San Jose, Calif.). Migration was followed either by a using a temperature-controlled fluorimeter (Fluostar Optima; BMG Labtechnologies, Offenburg, Germany) or by photomicrography of the underside of the opaque inserts. For the fluorometric assay, a standard curve was prepared with dye-labeled RS-MSCs and mMSCs placed in the bottom chambers of transwells with inserts. RS-MSCs or mMSCs at a concentration of 1×10⁵ cells/mL in 300 μL of serum free medium were placed in the upper chamber. The bottom chambers were loaded with 1 mL serum-free medium containing 1×10⁵ neural stem cells obtained from mouse brain of postnatal days 2 to 3, 50 ng/mL SDF-1 (R&D Systems, Minneapolis, Minn.), or 10 ng/mL Fractalkine (Chemicon). For neutralization studies, MSCs were incubated for 30 minutes at room temperature with 10 μg/mL anti-human CXCR4 (clone 12G5; R&D Systems) or 5 μg/mL anti-human CX3CR1 (clone 2A9-1; MBL, Woburn, Mass.) before seeding. After loading both chambers, the transwells were incubated at 37° C. for 16 hours. Values obtained with the fluorometric assay were confirmed by photomicroscopy using a 20× objective of the underside of the inserts and counting cells in 2 random fields per filter. Alternatively, the assays were by photomicrography alone.

### G. RT-PCR Assays

RNA was isolated from 1×10⁶ cells by the RNeasy RNA Isolation Kit (Qiagen, Valencia, Calif.) and 100 ng total RNA was used to perform reverse transcriptase (RT)-PCR assays with a commercial kit (M-MLV RT; Invitrogen). The samples were incubated at 37° C. for 50 minutes followed by 15 minutes at 70° C. to inactivate the RT. The cDNAs were amplified by PCR (Recombinant Taq DNA polymerase; Invitrogen) with 30 cycles at 94° C. for 30 seconds, 60° C. for 30 seconds, and 72° C. for 30 seconds.

TABLE 1
PCR Primers

| | | |
|---|---|---|
| Gene | Primer (5′→3′) | SEQ ID NO: |

| | | |
|---|---|---|
| mouse albumin | GAAAACCAGGCGACTATCT | SEQ ID NO:1 |
| forward | CCA | |
| albumin reverse | TGCACACTTCCTGGTCCTC | SEQ ID NO:2 |
| primer | A | |
| Alu forward | CATGGTGAAACCCCGTCTC | SEQ ID NO:3 |
| | TA | |
| Alu reverse | GCCTCAGCCTCCCGAGTAG | SEQ ID NO:4 |
| TaqMan probe | FAM-ATTAGCCGGGCGTGG | SEQ ID NO:5 |
| | TGGCG-TAMRA | |
| COL1A2 forward | CATCCACACACATGCACAG | SEQ ID NO:6 |
| primer | A | |
| COL1A2 reverse | TTTCCCCTTTGTTGTTTCC | SEQ ID NO:7 |
| primer | A | |
| SNP forward primer | GTAATCACAGCCTCCATGA | SEQ ID NO:8 |
| for the A/A allele | AATAGA | |
| SNP forward primer | GTAATCACAGCCTCCATGA | SEQ ID NO:9 |
| for the G/G allele | AATATG | |
| SNP reverse primer | ATAACATGGATTTTATCTA | SEQ ID NO:10 |
| for the A/A allele | AAATGTGT | |
| SNP reverse primer | ATAACATGGATTTTATCTA | SEQ ID NO:11 |
| for the G/G allele | AAATGTGC | |
| TaqMan probe 2 | FAMTGCCTAAAAAGCTATT | SEQ ID NO:12 |
| | GTGATGGAAAAGTGACAG | |
| | T-TAMRA | |
| CCR2 forward | CCAACGAGAGCGGTGAAGA | SEQ ID NO:13 |
| | AGTC | |
| CCR2 reverse | TCCGCCAAAATAACCGATG | SEQ ID NO:14 |
| | TGAT | |
| CXCR2 forward | CCGCCCCATGTGAACCAGA | SEQ ID NO:15 |
| | A | |
| CXCR2 reverse | AGGGCCAGGAGCAAGGACA | SEQ ID NO:16 |
| | GAC | |
| CX3CR1 forward | TCCTTCTGGTGGTCATCG | SEQ ID NO:17 |
| CX3CR1 reverse | TGTGCATTGGGTCCATCA | SEQ ID NO:18 |
| CXCR4 forward | GGTGGTCTATGTTGGCGTC | SEQ ID NO:19 |
| | T | |
| CXCR4 reverse | TGGAGTGTGACAGCTTGGA | SEQ ID NO:20 |
| | G | |
| CCR5 forward | CTGGCCATCTCTGACCTGT | SEQ ID NO:21 |
| | TTTTC | |
| CCR5 reverse | CAGCCCTGTGCCTCTTCTT | SEQ ID NO:22 |
| | CTCAT | |
| GAPDH forward | TCAACGGATTTGGTCGTAT | SEQ ID NO:23 |
| | TGGG | |
| GAPDH reverse | TGATTTTGGAGGGATCTCG | SEQ ID NO:24 |
| | C | |

### H. Real-Time RT-PCR Assays for CXCR4 and CX3CR1

Total RNA from RS-MSCs, mMSCs, and total MSCs was extracted (RNeasy Mini Kit; Qiagen) and reverse transcribed (M-MLV RT; Invitrogen). CDNA was analyzed by real-time PCR (ABI 7700 Sequence Detector; Applied Biosystems) using the same primers as for the RT-PCR assays and a DNA dye (SYBR Green PCR Reagents; Applied Biosystems). Reactions were incubated at 50° C. for 2 minutes, 95° C. for 10 minutes, and then 40 cycles at 95° C. for 15 seconds followed by 62° C. for 1 minute.

### I. Flow Cytometry of CXCR4 and CX3R1 Epitopes

RS-MSCs and mMSCs were isolated by FACS and about 17,500 cells plated in a 175-cm² dish. The cells were incubated in CCM for 5 days and lifted with 1 mM EDTA for 10 minutes at 37° C. About 1×10⁵ MSCs were stained by suspending the cells in 50 μL PBS containing 1% bovine serum albumin (BSA) and incubating for 30 minutes at 4° C. with 10 μg/mL mouse monoclonal anti-human CXCR4-PE (clone 12G5; R&D Systems) or anti-human CX3CR1-FITC (clone 2A9-1; MBL). Cells were analyzed by flow cytometry (FACScalibur; BD Biosciences) with CellQuest software.

J. Isolation and Characterization of RS-MSCs and mMSCs

RS-MSCs and mMSCs were separated from early passage low-density cultures of the MSCs by FACS on the basis of forward scatter (FS) and side scatter (SC) of light (FIG. 1). In initial reports (32), RS-MSCs were defined as the small population that clearly separated from the major body of the cells in plots of FS/SS. Subsequently, it was found that in order to obtain a large number of cells free of cellular debris, it was convenient to focus on a population of RS-MSCs that was recovered in the lower left quadrant of the distribution plot obtained by sorted unfractionated cultures of MSCs by FS/SS (33, 34). The cells defined as RS-MSCs by this criterion were slightly larger but similar to the original population of RS-MSCs in that they expanded rapidly with doubling times of 10 to 12 hours and readily differentiated in culture (32, 33). Also, up to 90% of the RS-MSCs generated single-cell-derived colonies in a clonogenic assay. However, this population of RS-MSCs became morphologically heterogeneous as colonies expand (32, 33).

The RS-MSCs were analyzed by re-assay of the cells by flow cytometry and cell cycle analysis (FIG. 1). Essentially all of the initial sort of RS-MSCs were again recovered as RS-MSCs. About 90% of the RS-MSCs were in G1 (FIGS. 1A and 1C). In contrast, the fraction of mMSCs contained small and variable amounts of cells with low FS/SS, apparently because of adherence of some of the small cells to the larger mMSCs (FIG. 1B). As expected, cell cycle assays indicated that the mMSCs were asynchronous (FIG. 1D). RS-MSCs and mMSCs isolated as shown in FIG. 1 were used in all subsequent experiments in this Example.

### K. Real-Time PCR-SNP Assay for Detection of Low Levels of Engraftment of MSCs

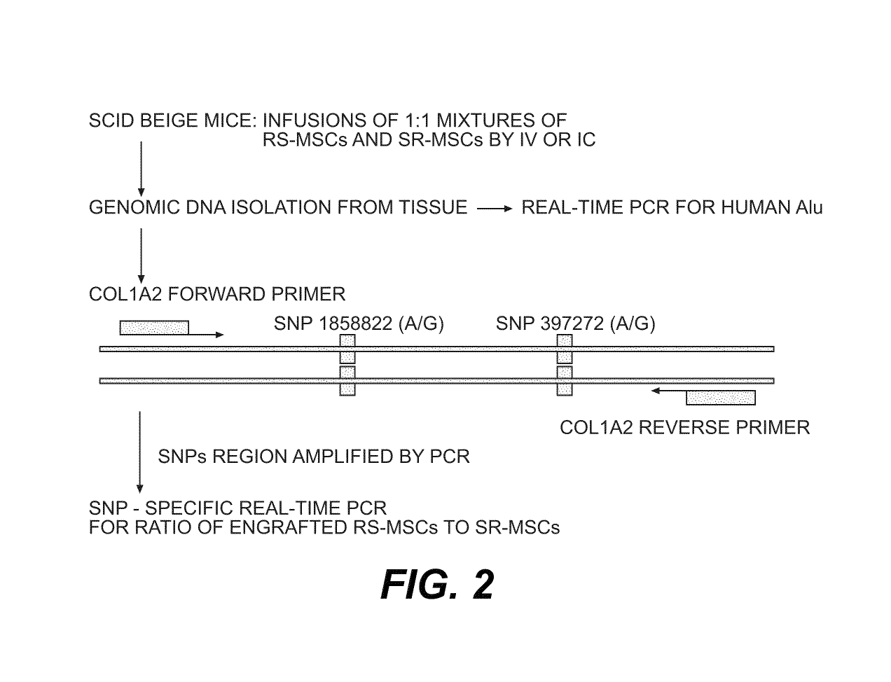

Preliminary experiments indicated that human MSCs infused into immunodeficient mice engrafted at very low levels. Two, sensitive, real-time PCR assays were used to compare the competitive engraftment of RS-MSCs and mMSCs (FIG. 2). One assay was for the highly repetitive human Alu sequences, to determine the total level of engraftment of human cells. Standard curves demonstrated that the assay detected 1 pg human DNA in samples containing 200 ng mouse DNA (not shown). The second assay was an SNP-based assay that was used to determine the ratio of RS-MSCs to mMSCs in tissues of mice after 1:1 mixtures of cells from 2 different donors were infused.

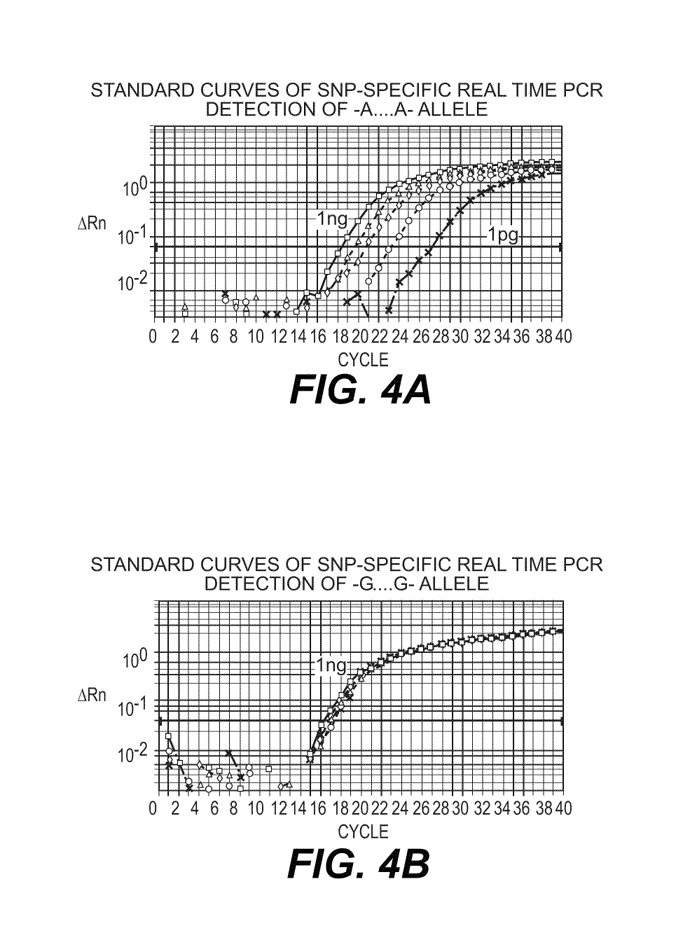

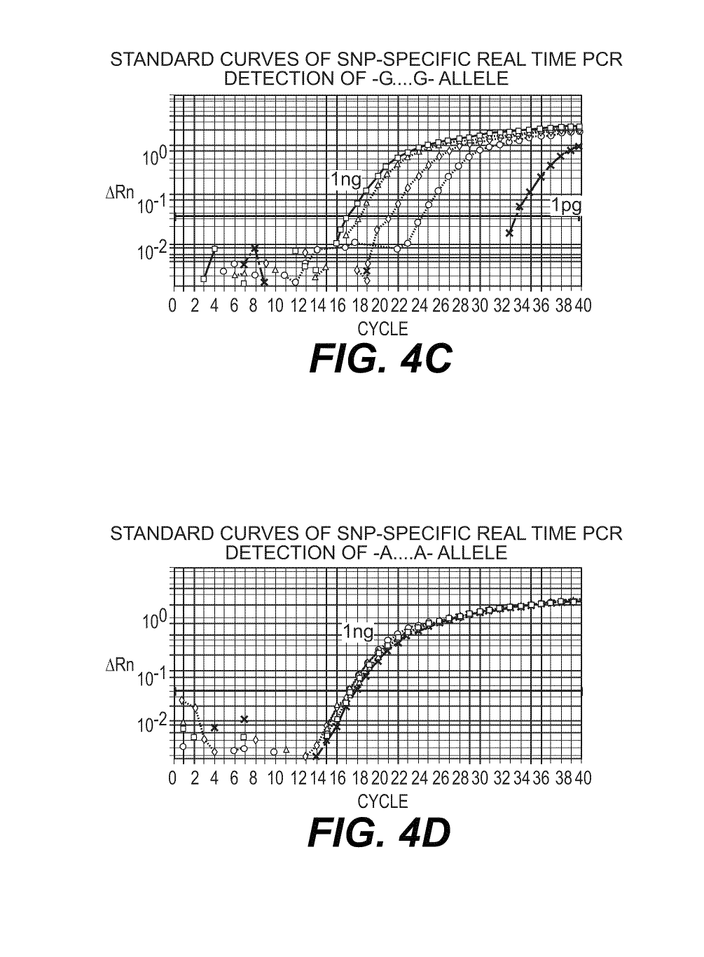

The first step of the SNP assay amplified a 630-bp region of the COL1A2 gene for the pro2 (I) chain of type I collagen (36) (FIG. 2) that contains 2 high-frequency G/A SNPs (Table 2 and FIG. 3). The second step of the SNP assay used nested primers to specifically amplify either the allele with 2 G-G SNPs or the allele with the A-A SNPs. Experiments to standardize the assay indicated that it specifically detected either 1 pg of the G-G allele or 1 pg of the A-A allele in samples that contained 1 ng of the alternative allele and 200 ng of mouse DNA (FIG. 4).

L. Competitive Engraftment of RS-MSCs and mMSCs after Intravenous Infusion

To assay the competitive engraftment of RS-MSCs and mMSCs, 1:1 mixtures of RS-MSCs and mMSCs from 2 human donors with different SNP alleles were infused intravenously into immunodeficient mice without marrow ablation or other preconditioning. Real-time PCR assays for the human Alu sequences demonstrated low and variable levels of the engraftment in various tissues (FIGS. 7A and 7B). The levels of engraftment varied from not detectable (<1 human cell per 200,000 mouse cells) to several thousand per lung, spleen, kidney, heart, and selective regions of brain. All of the mice assayed demonstrated a detectable level of engraftment in one or more of the organs assayed. However, none of the 8 mice assayed showed engraftment into all the organs examined. In the mouse with the highest detected levels (mouse no. 6 in FIG. 7A), the sum of all the organs assayed was about 78,000 human cells, or about 15% of the cells injected.

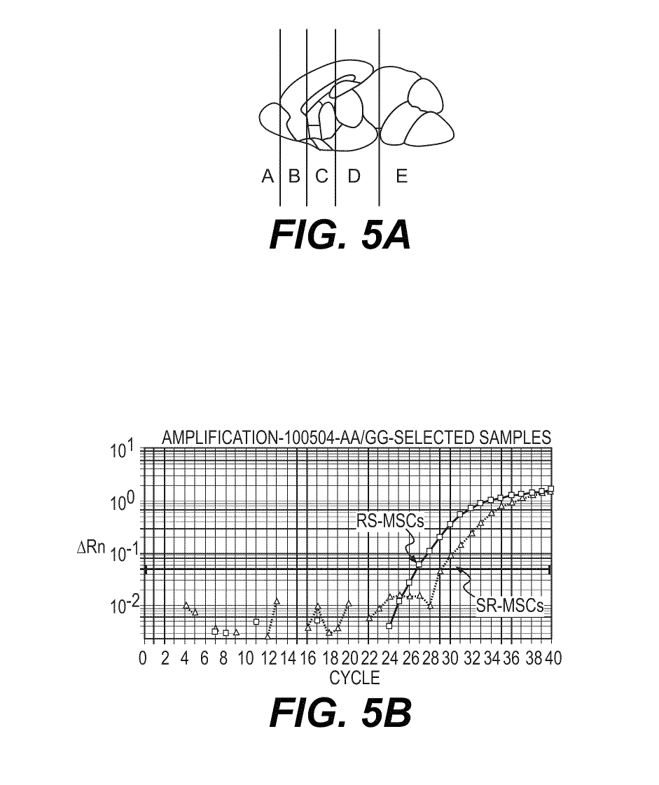

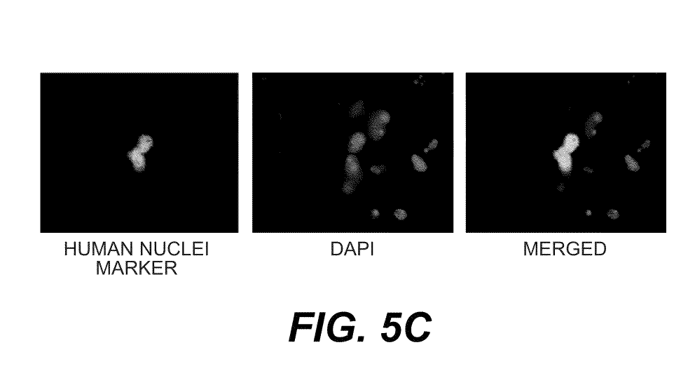

The allele-specific SNP assays were carried out on DNA samples that were identified by the assays for Alu sequences as containing at least 1 pg human DNA per 200 ng mouse DNA. The results of the allele-specific SNP assay indicated that with one exception (brain section D from mouse no. 5), the RS-MSCs engraft more efficiently than the mMSCs (FIGS. 7A and 7B; FIG. 5B). In several samples, the allele-specific SNP assay detected RS-MSCs but not mMSCs (denoted as ratio of >1000:1).

The presence of human cells in brain was confirmed by immunostaining of sections for the antibody for human nuclear specific protein (FIG. 5C). The number of human cells detected was too low to develop definite data on the expression of neural proteins by double immunostaining with antibodies to the human nuclei antigen and neural proteins without assaying a prohibitive number of brain sections.

M. Competitive Engraftment of RS-MSCs and mMSCs after Intracerebral Infusion

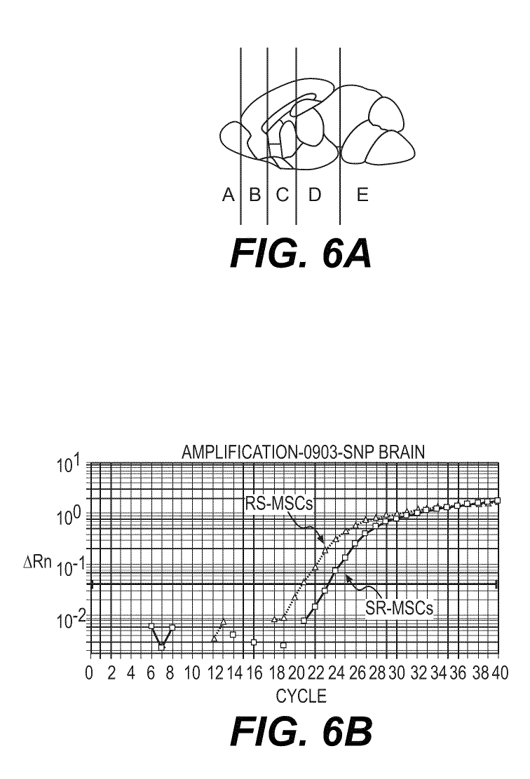

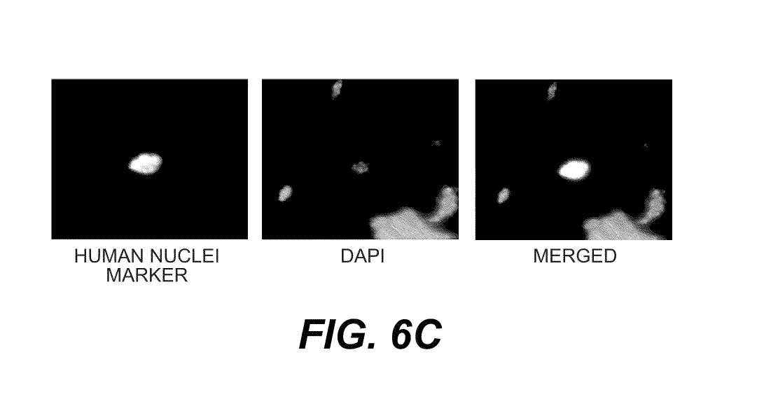

To further examine engraftment of the cells in brain, mixtures of RS-MSCs and mMSCs or RS-MSCs alone were injected directly into the hippocampus of the immunodeficient mice. After 2 weeks, human cells were detected by the assay for Alu sequences in brain section D containing the hippocampus in all 4 of the mice assayed (Table 3, FIG. 6A). In 2 mice, the human cells were also detected in the adjacent section containing the cerebellum, an observation probably explained by the fact that the bevel of the needle was directed caudally during the injection.

The allele-specific SNP assay again indicated that the RS-MSCs engrafted more efficiently in the 2 mice in which a 1:1 mixture of RS-MSCs and mMSCs were injected (ratios presented in footnotes in Table 3). The presence of human cells in the mouse brain was confirmed by antibody staining to a human-specific nuclear protein (FIG. 6C).

N. Comparison of RS-MSCs and mMSCs in Migration Assays to Neurospheres

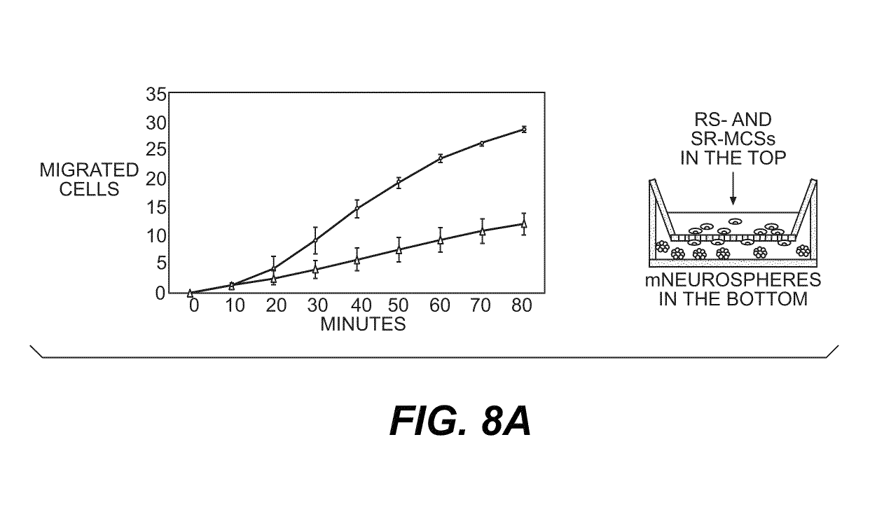

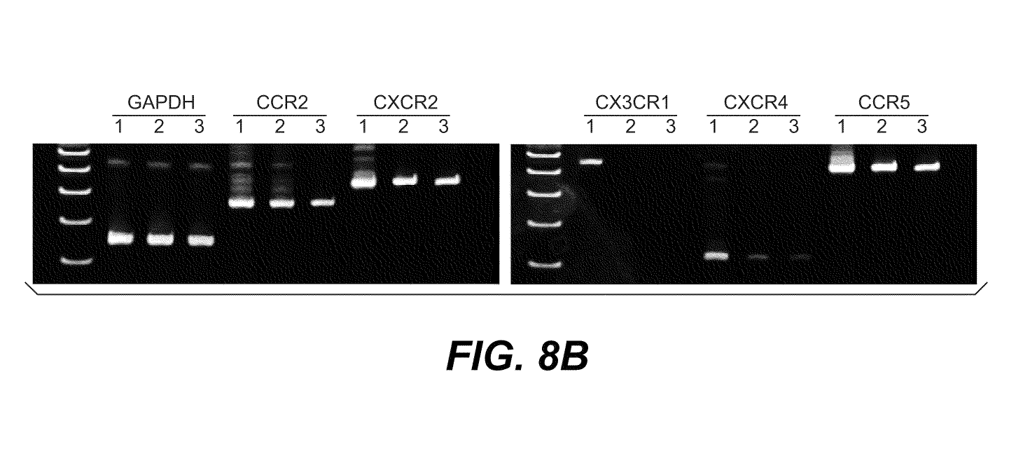

Because RS-MSCs were found to engraft more efficiently than mMSCs into the brain, the cells were compared in an in vitro migration assay with neurospheres. Murine neurospheres containing about 1×10⁵ neural stem cells were placed in the lower chamber of transwells and isolated RS-MSCs and mMSCs were placed in the upper chambers. The neurospheres increased the migration of both populations, but the RS-MSCs migrated more rapidly than the mMSCs (FIG. 8A).

O. RS-MSCs Express Higher Levels of CXCR4 and CXC3R1 than mMSCs

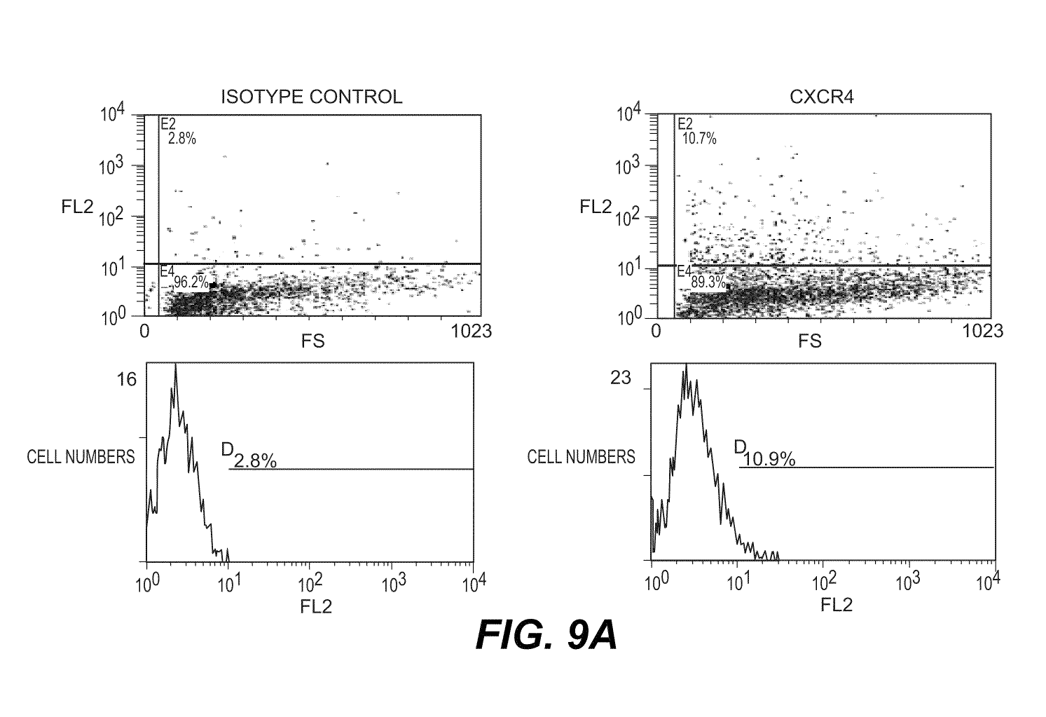

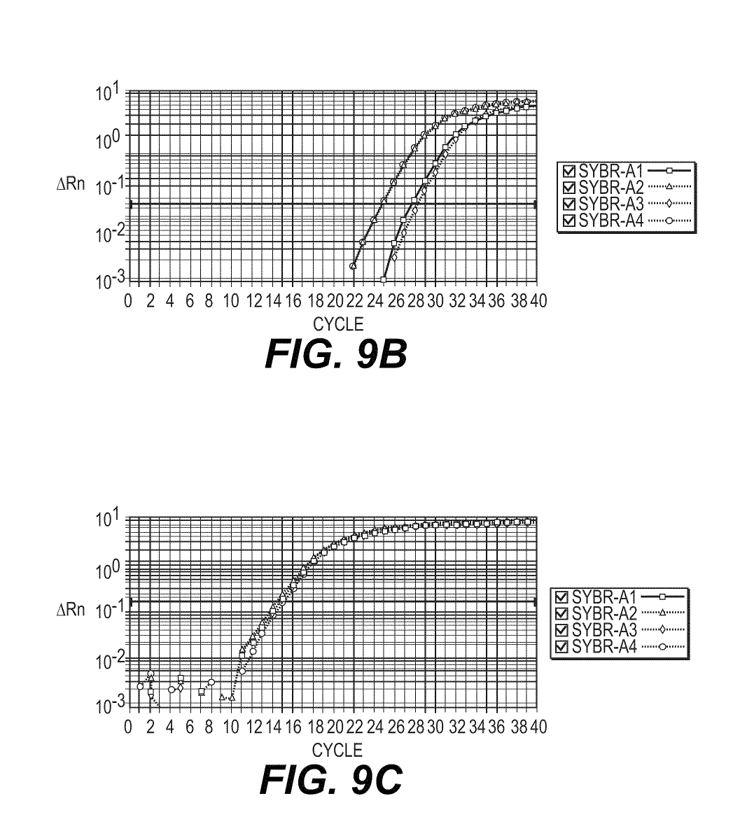

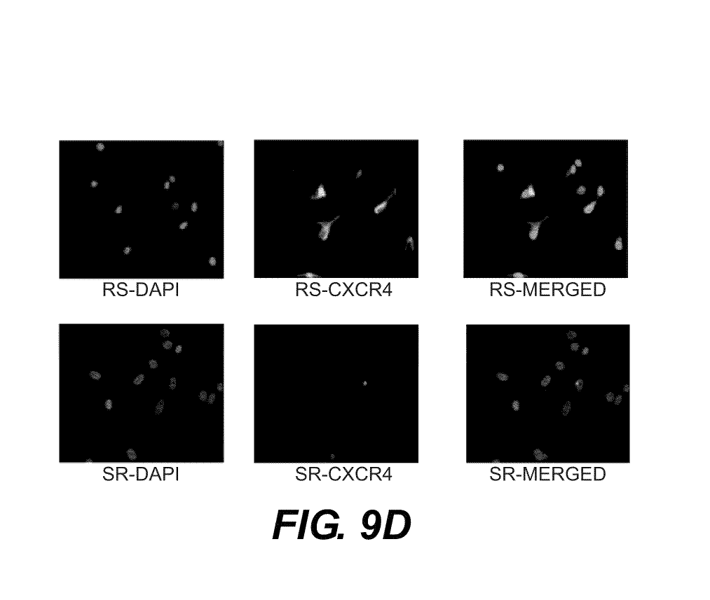

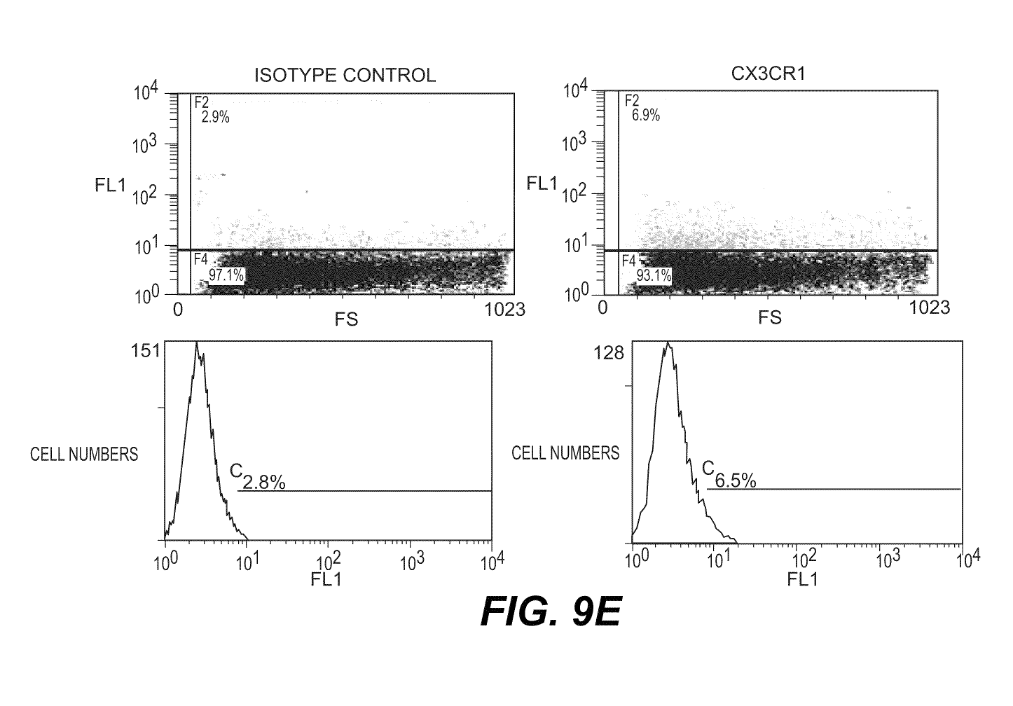

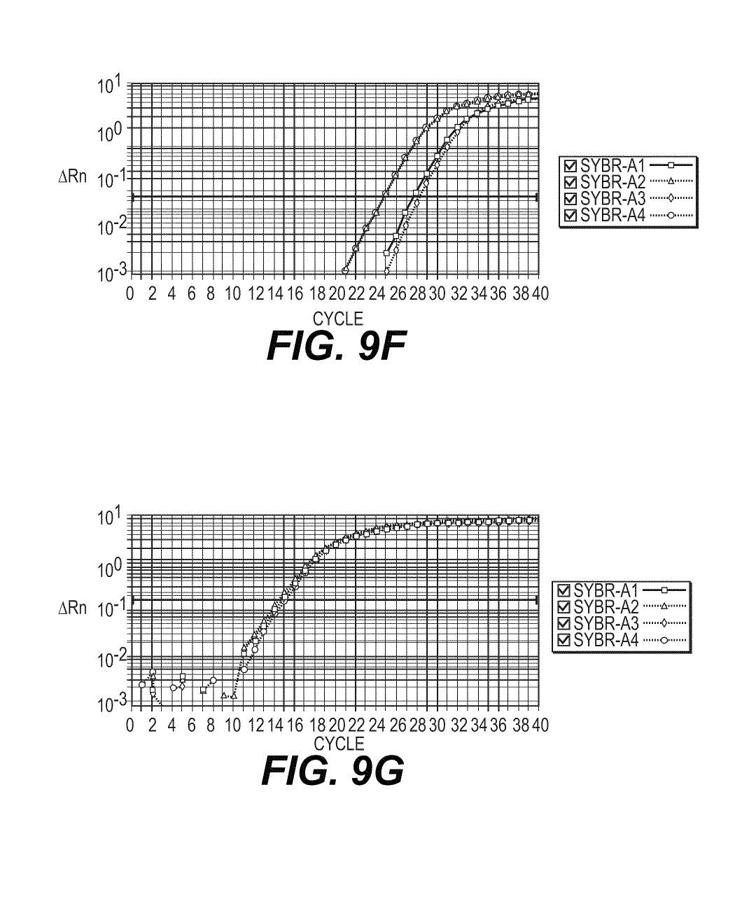

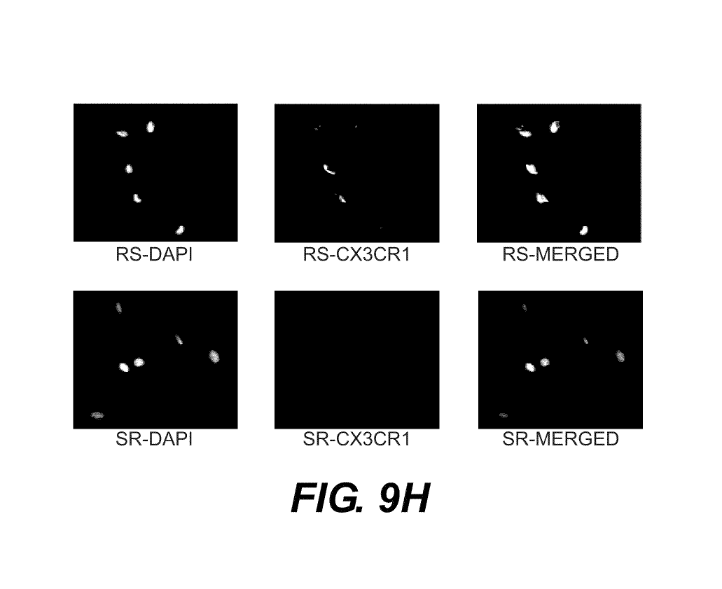

To identify molecular bases for the preferential engraftment and migration ability of RS-MSCs, the expression patterns for several chemokine receptors was compared in RS-MSCs and mMSCs. Semiquantitative PCR assays detected the receptors CCR2, CXCR2, and CCR5 in both RS-MSCs and mMSCs (FIG. 8B). However, only the RS-MSCs contained mRNA for CXCR4, the receptor for SDF-1, and CX3CR1, the receptor for fractalkine. Flow cytometry assays of unfractionated MSCs demonstrated that about 8% of the cells expressed CXCR4 as a surface epitope (FIG. 9A). The cells that expressed the receptor were cells with low FS, corresponding to RS-MSCs. Real-time RT-PCR assays demonstrated that the level of mRNA for CXCR4 was about 10-fold higher in RS-MSCs than in mMSCs (FIG. 9B). The high expression of CXCR4 in RS-MSCs was confirmed by isolating RS-MSCs by FACS, culturing them for 1 day in chambered slides, and staining them with an anti-CXCR4 antibody (FIG. 9D).

Similar assays demonstrated that about 3% of unfractionated MSCs expressed CX3CR1, the receptor for fractalkine (FIG. 9E). The receptor was also found primarily on the cells with low FS of light corresponding to RS-MSCs. Real-time PCR assays indicated that the levels of mRNA for CX3CR1 were about 10-fold higher in RS-MSCs than in mMSCs (FIGS. 9F-G). The higher expression of CX3CR1 by RS-MSCs was confirmed by immunostaining of RS-MSCs isolated by FACS (FIG. 9H).

P. Comparison of RS-MSCs and mMSCs in Migration Assays

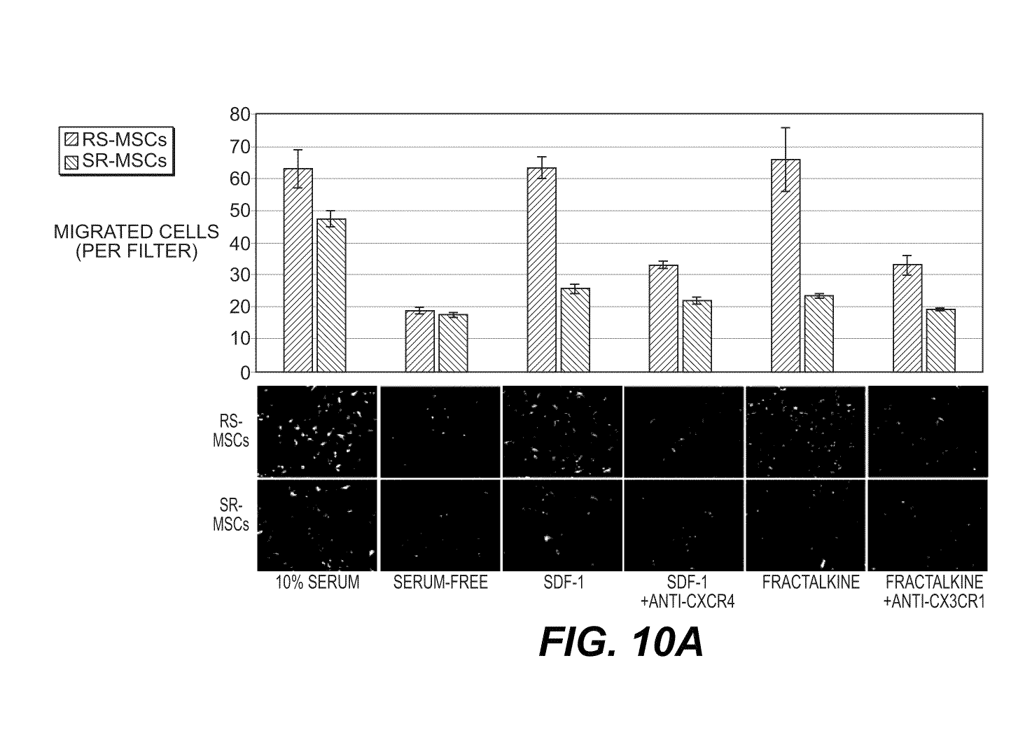

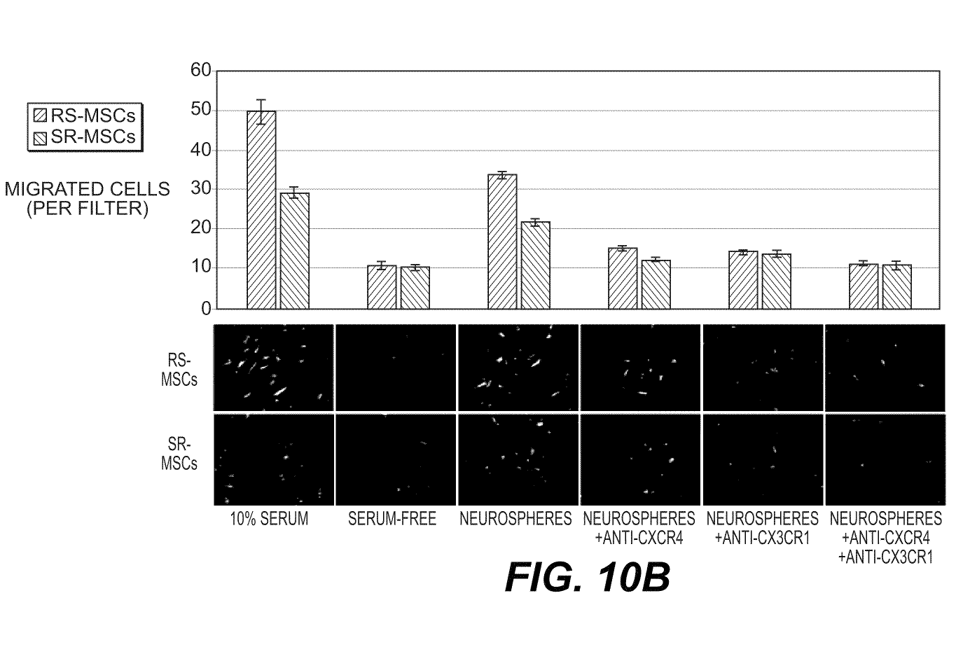

We then compared the migration of 2 subpopulations of MSCs to SDF-1, the ligand for CXCR4, and fractalkine, the ligand for CX3CR1. SDF-1 increased the migration of RS-MSCs more than mMSCs relative to serum-free controls (FIG. 10A). Antibodies to CXCR4 decreased the SDF-1-stimulated migration. Similar results were obtained with fractalkine and antibodies to CX3CR1 (FIG. 10A).

The effects of antibodies to CXCR4 and CX3CR1 on the neurosphere-induced migration of the cells were also examined (FIG. 10B). Antibodies to CXCR4 decreased the induced migration of RS-MSCs and mMSCs by about 40%. Antibodies to CX3CR1 had a similar effect and produced about a 40% decrease in migration of both cell types. The effects of the two antibodies were only partially additive. Therefore, the results indicated the neurosphere-induced migration of the cells was partially but not completely dependent on CXCR4 and CX3CR1 pathways.

The small size of RS-MSCs may explain both the increased migration observed in the in vitro assays and their more efficient penetration into tissues in vivo. However, the more rapid migration of RS-MSCs was not explained by the smaller size of the cells, since RS-MSCs and mMSCs migrated at the same or similar rates when medium containing 10% FCS was placed in the lower well or in the presence of blocking antibodies (FIG. 10A-B).

TABLE 2
SNPs used as specific markers for donor cells

| | dbSNP rs# | | | |
|---|---|---|---|---|
| Contig position | cluster id* | heterozygosity | SNP | Function |
| 19266969 | rs1858822 | 0.485 | G/A | Intron(COL1A2) |
| 19267158 | rs397272 | 0.499 | G/A | Intron(COL1A2) |

*rs#: reference SNP

TABLE 3
Engraftment after intracranial injections: human cells
detected by real-time PCR for human Alu sequences

| | | Brain section | | | | |
|---|---|---|---|---|---|---|
| Mouse no. | Injected subpopulation | A | B | C | D | E |
| 1 | RS-MSCs + mMSCSs | ND | ND | ND | 1489 ± 70.2* | ND |
| 2 | RS-MSCs + mMSCSs | ND | ND | ND | 849 ± 80.5^{†} | 360 ± 7.5 |
| 3 | RS-MSCs only | ND | ND | ND | 1646 ± 71.8 | ND |
| 4 | RS-MSCs only | ND | ND | ND | 689 ± 56.2 | 623 ± 44.1 |

Values indicate the average number of human MSCs ± SD. All mice were measured at 2 weeks after injection. See FIG. 7B for expression of data as percent of infused MSCs.
A indicates olfactory bulb; B, olfactory cortex; C, striatum; D, hippocampus; E, cerebellum; ND, not detected with assay sensitivity to 1 human cell per 2 × 10⁵ mouse cells.
*Ratio of RS-MSCs to mMSCs by SNP assay 10:1
^{†}Ratio of RS-MSCs to mMSCs by SNP assay greater than 1000:1

TABLE 4
Engraftment in the tissues after tail vein injections: human cells detected by real-time PCR for human Alu sequences.

| | Weeks | | | | | | | | | | | |
|---|---|---|---|---|---|---|---|---|---|---|---|---|
| Mouse | after | Brain section | | | | | | | | | | |
| no. | injection | A | B | C | D | E | BM | Heart | Liver | Kidney | Spleen | Lung |
| 1 | 1 | ND | 180 ± | 246 ± | 157 ± | 1,562 ± | ND | 377 ± | ND | 3781 ± | 2225 ± | 5656 ± |
| | | | 9.8 | 24.0 | 26.7 | 56.2* | | 57.2 | | 45.3 | 121.2* | 85.2 |
| 2 | 1 | ND | 88 ± | ND | ND | ND | + | ND | ND | 235 ± | ND | 1756 ± |
| | | | 12.3 | | | | | | | 58.2 | | 45.6 |
| 3 | 3 | 4044 ± | ND | ND | 480 ± | ND | + | 380 ± | ND | ND | 3715 ± | ND |
| | | 54.3* | | | 41.6^{†} | | | 24.5 | | | 81.9* | |
| 4 | 3 | ND | 130 ± | ND | 113 ± | ND | + | 1882 ± | 15,246 ± | ND | ND | 1110 ± |
| | | | 59.8 | | 2.3 | | | 14.2* | 75.6 | | | 75.6 |
| 5 | 3 | ND | ND | ND | 545 ± | 265 ± | ND | 364 ± | ND | ND | 1466 ± | ND |
| | | | | | 98.3^{‡} | 45.3 | | 9.2 | | | 67.3 | |
| 6 | 3 | ND | ND | ND | ND | 75,086 ± | + | 320 ± | ND | ND | 1427 ± | 1130 ± |
| | | | | | | 98.6* | | 13.9 | | | 8.9 | 56.8 |
| 7 | 5 | ND | ND | 1096 ± | 248 ± | ND | ND | 924 ± | ND | ND | 1954 ± | ND |
| | | | | 84.2 | 45.3 | | | 51.0 | | | 54.8 | |
| 8 | 5 | ND | 297 ± | ND | 530 ± | 780 ± | ND | 372 ± | ND | ND | ND | 1300 ± |
| | | | 26.3* | | 49.6* | 87.6 | | 5.6 | | | | 46.9* |

Real-time PCR assays for human Alu sequences are expressed as number of human cells per organ.
Ratios of RS-MSCs to mMSCs as assayed by the competitive SNP assay are presented in the symbolled footnotes.
Values indicate the average number of human MSCs ± SD.
A indicates olfactory bulb; B, olfactory cortex; C, striatum; D, hippocampus; E, cerebellum; ND, not detected with assay sensitivity to 1 human cell per 2 × 10⁵ mouse cells; +, Alu sequences detected.
*Ratio of RS-MSCs to mMSCs by SNP assay greater than 1000:1
^{†}Ratio of RS-MSCs to mMSCs by SNP assay 10:1
^{‡}Ratio of RS-MSCs to mMSCs by SNP assay less than 1:1000

### REFERENCES

1. A. J. Friedenstein et al., Cell Tissue Kinet. 3, 393-403 (1970).
2. H. Castro-Malaspina et al., Blood 56, 289-301 (1980).
3. T. S. Mets et al., Mech. Ageing Dev. 16, 81-89 (1981).
4. A. H. Piersma et al., Exp. Hematol. 13, 237-243 (1985).
5. M. Owen et al., Ciba Found Symp. 136, 42-60 (1988).
6. A. I. Caplan, J. Orthoped. Res. 9, 640-650 (1991).
7. B. R. Clark et al., Ann. N.Y. Acad. Sci. 770, 70-78 (1995).
8. N. N. Beresford et al., J. Cell Sci. 102, 341-351 (1992).
9. S. Wakitani et al., Muscle Nerve 18, 1417-1426 (1995).
10. R. F. Pereira et al., Proc. Natl. Acad. Sci. U.S.A. 92, 4857-4861 (1995).
11. S. A. Kuznetsov et al., Br. J. Haematol. 97, 561-570 (1997).
12. S. P. Bruder et al., J. Cell Biochem. 64, 287-294 (1997).
13. D. J. Prockop, Science 276, 71-74 (1997).
14. R. F. Pereira et al., Proc. Natl. Acad. Sci. U.S.A. 95, 7294-7299 (1998).
15. B. Johnstone et al., Exp. Cell Res. 238, 265-272 (1998).
16. G. Ferrari et al., Science. 279, 1528-1530 (1998).
17. S. A. Azizi et al., Proc Natl Acad Sci USA. 95, 3908-3913 (1998).
18. M. F. Pittenger et al., Science 284, 143-147 (1999).
19. S. K. Nilsson et al., J Exp. Med. 189, 729-734 (1999).
20. Z. Hou et al., Proc Natl Acad Sci USA. 96, 7294-7299 (1999).
21. C. M. DiGirolamo et al., Br. J. Haematol. 107, 275-281 (1999).
22. E. M. Horwitz et al., Nat. Med. 5, 309-313 (1999).
23. G. C. Kopen et al., Proc Natl Acad Sci USA. 96, 10711-10716 (1999).
24. A. I. Caplan, Clin. Orthoped. 379, 567-570 (2000).
25. D. Colter et al., Proc. Natl. Acad. Sci. 97, 3213-3218 (2000).
26. M. Chopp et al., Neuroreport. 11, 3001-3005 (2000).
27. D. Woodbury et al., J. Neuroscience Res. 61, 364-370 (2000).
28. K. W. Liechty et al., Nat Med. 6, 1282-1286 (2000).
29. D. S. Krause et al., Cell. 105, 369-377 (2001).
30. Y. Jiang et al., Nature. 418, 41-49 (2002).
31. A. J. Wagers et al., Cell. 116, 639-648 (2004).
32. D. C. Colter et al., Proc Natl Acad Sci USA. 98, 7841-7845 (2001).
33. J. R. Smith et al., Stem Cells. 22, 823-831 (2004).
34. I. Sekiya et al., Stem Cells. 20, 530-541 (2002).
35. C. McBride et al., Cytotherapy. 5, 7-18 (2003).
36. J. Korkko et al., Am J Hum Genet. 62, 98-110 (1998).
37. F. Maas et al., Leukemia. 17, 621-629 (2003).
38. D. N. Koffon et al., Development. 128, 5181-5188 (2001).
39. National Center for Biotechnology Information. Entrez Genome. http://www.ncbi.nlm.nih.gov/entrez/query.fcgi?db=Genome. Accessed Jun. 1, 2005.
40. U.S. Pat. No. 7,056,738.
41. Lennon et al., Exp. Cell Res. 219, 211-222 (1995)
42. Toma et al., Circulation 105, 93-98 (2002)
43. Peterson et. al., Science 284, 1168-1170 (1999).
44. Sanchez-Ramos et. al., Exp. Neurol. 164, 247-256 (2002).
45. Deng et. al., Biochem. Biophys. Res. Common. 282, 148-152 (2001).
46. Schwartz et. al., Hum Gene Ther. 10, 2539-2549 (1999).
47. Wu et. al., J Neurosc. Res. 72, 383 (2003).
48. Tomita et. al., Circulation 100, 247 (1999).
49. Shake et. al., Ann. Thorac. Surg. 73, 1919 (2002).
50. Horwitz et. al., Blood 97, 1227 (2001).
51. Horwitz et. al., Proc. Natl. Acad. Sci. USA 99, 8932 (2002).
52. Frassoni et. al., Int. Society for Cell Therapy SA006 (abstract) (2002).
53. Koc et. al., J. Clin. Oncol. 18, 307 (2000).
54. Okamoto et. al., Nature Med. 8, 1101-1017 (2002).
55. Erices et. al., Br. J. Haematol. 109, 235-42 (2000).
56. Djouad et. al., Arthritis Res. & Ther. 7, R1304-R1315 (2005).
57. Gronthos et. al., J. Hematother. 5, 15-23 (1996).

What is claimed is:

1. A method for enhancing engraftment of MSCs in an individual in need thereof comprising administering to the individual a population of MSCs enriched for RS-MSCs that express at least one of the polypeptides selected from CXCR4 and CX3CR1, wherein the MSCs are administered in an amount effective to promote engraftment of said MSCs in said individual.

2. The method of claim 1, wherein the MSCs are administered by intravenous injection, injection directly to the site of intended activity, or by infusion.

3. The method of claim 1, wherein the MSCs are autologous.

4. The method of claim 1, wherein the MSCs are allogeneic.

5. The method of claim 1, wherein the MSCs are HLA compatible with the individual.

6. The method of claim 1, wherein the MSCs are isolated from a tissue selected from the group consisting of bone marrow, peripheral blood, umbilical cord blood, and synovial membrane.

7. The method of claim 6, wherein the MSCs are isolated from bone marrow.

8. The method of claim 1, wherein the MSCs express CXCR4 and CX3CR1.


SEQUENCE LISTING (25 sequences)

SEQ ID NO: 1 - DNA, 22 bp, Artificial Sequence
  Description of Artificial Sequence Synthetic primer
gaaaaccaggcgactatctcca

SEQ ID NO: 2 - DNA, 20 bp, Artificial Sequence
  Description of Artificial Sequence Synthetic primer
tgcacacttcctggtcctca

SEQ ID NO: 3 - DNA, 21 bp, Artificial Sequence
  Description of Artificial Sequence Synthetic primer
catggtgaaaccccgtctcta

SEQ ID NO: 4 - DNA, 19 bp, Artificial Sequence
  Description of Artificial Sequence Synthetic primer
gcctcagcctcccgagtag

SEQ ID NO: 5 - DNA, 20 bp, Artificial Sequence
  Description of Artificial Sequence Synthetic probe
attagccgggcgtggtggcg

SEQ ID NO: 6 - DNA, 20 bp, Artificial Sequence
  Description of Artificial Sequence Synthetic primer
catccacacacatgcacaga

SEQ ID NO: 7 - DNA, 20 bp, Artificial Sequence
  Description of Artificial Sequence Synthetic primer
tttcccctttgttgtttcca

SEQ ID NO: 8 - DNA, 25 bp, Artificial Sequence
  Description of Artificial Sequence Synthetic primer
gtaatcacagcctccatgaaataga

SEQ ID NO: 9 - DNA, 25 bp, Artificial Sequence
  Description of Artificial Sequence Synthetic primer
gtaatcacagcctccatgaaatatg

SEQ ID NO: 10 - DNA, 27 bp, Artificial Sequence
  Description of Artificial Sequence Synthetic primer
ataacatggattttatctaaaatgtgt

SEQ ID NO: 11 - DNA, 27 bp, Artificial Sequence
  Description of Artificial Sequence Synthetic primer
ataacatggattttatctaaaatgtgc

SEQ ID NO: 12 - DNA, 35 bp, Artificial Sequence
  Description of Artificial Sequence Synthetic probe
tgcctaaaaagctattgtgatggaaaagtgacagt

SEQ ID NO: 13 - DNA, 23 bp, Artificial Sequence
  Description of Artificial Sequence Synthetic primer
ccaacgagagcggtgaagaagtc

SEQ ID NO: 14 - DNA, 23 bp, Artificial Sequence
  Description of Artificial Sequence Synthetic primer
tccgccaaaataaccgatgtgat

SEQ ID NO: 15 - DNA, 20 bp, Artificial Sequence
  Description of Artificial Sequence Synthetic primer
ccgccccatgtgaaccagaa

SEQ ID NO: 16 - DNA, 22 bp, Artificial Sequence
  Description of Artificial Sequence Synthetic primer
agggccaggagcaaggacagac

SEQ ID NO: 17 - DNA, 18 bp, Artificial Sequence
  Description of Artificial Sequence Synthetic primer
tccttctggtggtcatcg

SEQ ID NO: 18 - DNA, 18 bp, Artificial Sequence
  Description of Artificial Sequence Synthetic primer
tgtgcattgggtccatca

SEQ ID NO: 19 - DNA, 20 bp, Artificial Sequence
  Description of Artificial Sequence Synthetic primer
ggtggtctatgttggcgtct

SEQ ID NO: 20 - DNA, 20 bp, Artificial Sequence
  Description of Artificial Sequence Synthetic primer
tggagtgtgacagcttggag

SEQ ID NO: 21 - DNA, 24 bp, Artificial Sequence
  Description of Artificial Sequence Synthetic primer
ctggccatctctgacctgtttttc

SEQ ID NO: 22 - DNA, 24 bp, Artificial Sequence
  Description of Artificial Sequence Synthetic primer
cagccctgtgcctcttcttctcat

SEQ ID NO: 23 - DNA, 23 bp, Artificial Sequence
  Description of Artificial Sequence Synthetic primer
tcaacggatttggtcgtattggg

SEQ ID NO: 24 - DNA, 20 bp, Artificial Sequence
  Description of Artificial Sequence Synthetic primer
tgattttggagggatctcgc

SEQ ID NO: 25 - DNA, 300 bp, Homo sapiens
taactataaaaccccacagggttcttctctgaattaatgagtaatcacagcctccatgaaatacrctacattttatgtaaatgaaattgttgcaaatacatgaaaaaataaatataattagaaattcatgatgtcaaagaaaattattttttaatgtatgcctaaaaagctattgtgatggaaaagtgacagtttcttttaatgtcagagcaatttctaaaaccaaatgaataattcttataattaaaatgacrtacattttagataaaatccatgttatttcactctaggcattaatac